(19) United States
(12) Patent Application Publication
(10) Pub. No.: US 2026/0224525 A1
(43) Pub. Date: Aug. 6, 2026

(54) PARENTERAL COMPOSITIONS OF ZILEUTON AND METHODS OF USE

(71) Applicant: University of Houston System, Houston, TX (US)

(72) Inventors: Vincent Tam, Houston, TX (US); Xinli Liu, Houston, TX (US); Cole Hudson, Houston, TX (US); Yongqi Xiao, Houston, TX (US)

(21) Appl. No.: 19/576,709

(22) Filed: Mar. 24, 2026

Related U.S. Application Data
(63) Continuation-in-part of application No. 19/105,589, filed on Aug. 23, 2023.
(60) Provisional application No. 63/373,275, filed on Aug. 23, 2022.

(51) Int. Cl. A61K 31/381; A61K 9/00; A61K 9/08; A61K 9/51; A61K 31/7036; A61K 38/12; A61K 47/10; A61K 47/20; A61K 47/26; A61P 13/12

(52) U.S. Cl. CPC A61K 31/381; A61K 9/0019; A61K 9/08; A61K 9/5123; A61K 9/5161; A61K 31/7036; A61K 38/12; A61K 47/10; A61K 47/20; A61K 47/26; A61P 13/12

(57) ABSTRACT

Provided herein are compositions containing zileuton formulated for parenteral administration and/or extended use. Methods of prevention and treatment are directed to subjects who may benefit from extended use of zileuton to provide renal protective effects.

### CROSS REFERENCE TO RELATED APPLICATIONS

The present patent application is a continuation-in-part of U.S. patent application Ser. No. 19/105,589, filed Feb. 21, 2025, which is a national phase application of PCT/US2023/072771, filed Aug. 23, 2023, which claims the benefit of and the priority to U.S. Provisional Patent Application No. 63/373,275, filed Aug. 23, 2022, each of which is incorporated by reference herein in its entirety.

### TECHNICAL FIELD

The disclosure relates to parenteral compositions of zileuton and methods of use of these compositions for treatment of disease.

### BACKGROUND

Zileuton is a 5-lipoxygenase inhibitor that inhibits the formation of leukotrienes, such as LTB4, LTC4, LTD4, and LTE4. Zileuton is approved by the FDA (under the trade name Zyflo®) for the prophylaxis and chronic treatment of asthma in adults and children twelve (12) years of age and older. For this indication, zileuton is commercially formulated as tablets for oral administration (for example regular, film-coated, or extended release tablets). In addition to the indication of zileuton to treat or prevent asthma, studies support the benefits of zileuton to treat other diseases or conditions.

### SUMMARY

Accordingly, Applicant has recognized a need for a pharmaceutical formulation of zileuton suitable for the parenteral route of drug administration (for example as an intravenous infusion). These compositions may be used for treating subjects for whom the oral route of administration is not a viable or reliable option or for treating diseases or conditions for which parenteral administration is suitable. The present disclosure provides formulations, compositions, kits, methods of preparation, and methods of treatment with zileuton formulated for parenteral administration. Methods of treating diseases or conditions that may benefit from extended use of zileuton for renal protective effects, for example infection and cancer for which nephrotoxicity-inducing treatment is used, are also provided. Additional pharmacological properties of zileuton were identified, which can be exerted through extended use. For example, preliminary studies in experimental animals support the ability of zileuton to reduce nephrotoxicity associated with selected antibiotics (for example, polymyxins and aminoglycosides). The subject population that may benefit from extended use of zileuton includes subjects with acute and serious infections, such as critically ill subjects, for whom the oral route of administration is not a viable or reliable option, or subjects who have diseases or conditions for treatment of which parenteral administration of zileuton is more suitable than oral administration.

One aspect of the present disclosure provides a pharmaceutical composition containing a therapeutically effective amount of zileuton and a biologically acceptable excipient formulated for parenteral administration. The pharmaceutical composition can be used for preventing, delaying the onset of, or treating acute kidney injury (AKI) or nephrotoxicity. In certain examples, the excipient is polyethylene glycol (PEG). The PEG component can include PEG with a molecular weight ranging from about 350 to about 650 Daltons, such as PEG 400, PEG 500, PEG 600, and PEG 400-600. Excipients can include water, saline, propylene glycol, glycerin, ethanol, sorbitol, triacetin, polyoxyethylated glycerides, polyoxyethylated oleic glycerides, hydroxypropyl-β-cyclodextrin, a surfactant, or any combinations thereof.

In certain examples, the excipient contains ethanol, PEG 400, and water, wherein ethanol is about 10% by volume and PEG 400 is about 25% to about 40% by volume, with water as the balance. In certain examples, the excipient contains ethanol, PEG 400 , and water in 1:4:5 or 10%:40%:50% by volume. In certain examples, the excipient contains about 10 vol. % ethanol, about 40 vol. % PEG 400 , and about 50 vol. % water. In certain examples, the excipient contains ethanol, PEG 400, and water in 10%:25%:65% by volume. In certain examples, the excipient contains about 10 vol. % ethanol, about 25 vol. % PEG 400, and about 65 vol. % water.

In one aspect, the present disclosure provides a nanoparticle-based composition containing a therapeutically effective amount of zileuton and a nanoparticle, wherein the parenteral composition is formulated for renal protection against a nephrotoxicity-inducing agent. In certain examples, the nanoparticle contains polysaccharide, a pegylated lipid, and/or phosphate counter ions. In certain examples, the nanoparticle contains a mean hydrodynamic diameter ranging from about 50 nanometers (nm) to about 300 nm and a near neutral charge. In certain examples, the nanoparticle contains a mean hydrodynamic diameter of about 280 nm and a mean zeta potential of about −3 mV. In certain examples, the nanoparticle contains zileuton in an inner cavity of the nanoparticle. In certain examples, the composition accumulates in proximal tubule epithelia when administered to subjects.

In certain examples, the parenteral compositions or nanoparticle-based compositions provided herein contain about 1 mg/mL zileuton. In certain examples, the zileuton degrades less than 10% over 140 hours to 150 hours at −20 degrees centigrade (° C.), 4° C., or 22° C. in the pharmaceutical composition.

In certain examples, the pharmaceutical composition is used in preventing, delaying the onset of, or otherwise treating AKI or nephrotoxicity in a subject. Provided herein is a method of preventing, delaying the onset of, or treating AKI or nephrotoxicity in a subject by administering (such as parenterally administering) a pharmaceutical composition containing a therapeutically effective amount of zileuton and a biologically acceptable excipient formulated for parenteral administration.

In certain examples, the AKI or nephrotoxicity is induced by a drug, such as an antibiotic, an anti-cancer drug (chemotherapy), or a diagnostic contrast agent. Administration of the pharmaceutical composition provided herein can have a nephroprotective effect against the nephrotoxicity-inducing agent or the AKI-inducing agent.

In certain examples, the nephrotoxicity-inducing agent administered to the subjects contains an antibiotic or an anti-cancer drug. In certain examples, the antibiotic can be one or more of amikacin, tobramycin, netilmicin, streptomycin, arbekacin, plazomicin, neomycin, kanamycin, paromomycin, gentamicin, bacitracin, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815, and octapeptin C4. In certain examples, the anti-cancer drug comprises ifosfamide, ipilimumab, pembrolizumab, and/or nivolumab.

In certain examples, the pharmaceutical composition is administered prior to or contemporaneously with administration of the drug.

In certain examples, AKI or nephrotoxicity is induced by renal ischemia. In certain examples, the renal ischemia is associated with ischemic shock, surgery, or transplant organ procurement. In certain examples, the pharmaceutical composition is administered prophylactically before an anticipated ischemic event or therapeutically after an ischemic event. The ischemic event can be ischemic shock, surgery (such as cardiac surgery), or transplant organ procurement. In embodiments, the pharmaceutical composition is administered to the subject at about 10 hours and about 2 hours prior to an anticipated ischemic event, and 6 hours following the event.

In certain examples, the pharmaceutical composition provided herein is administered intravenously, intramuscularly, subcutaneously, intraperitoneally, intranasally, or intrathecally to a subject. In certain examples, parenteral administration is selected for subjects with severe infection, critical illness, or subjects who are otherwise unable to tolerate an oral formulation.

Embodiments include methods of preparing a parenteral composition by combining an effective amount of zileuton with a biologically acceptable excipient for parenteral administration. Embodiments also include methods of administering a parenteral composition containing an effective amount of zileuton to a subject. In certain examples, the subject has a severe infection, is critically ill, or is otherwise unable to handle an oral formulation.

Embodiments include methods of treating a subject with infection by administering a parenteral composition containing an effective amount of a zileuton-containing composition and an effective amount of a nephrotoxicity-inducing antibiotic. The antibiotics can be one or more of amikacin, tobramycin, netilmicin, streptomycin, arbekacin, plazomicin, neomycin, kanamycin, paromomycin, gentamicin, bacitracin, polymyxin B, colistin, amphotericin B, tetracyclines, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815, and octapeptin C4. In certain examples, the effective amount of zileuton can range from about 0.5 mg/kg to about 200 mg/kg.

Embodiments include methods of treating a subject with cancer by administering a parenteral composition containing an effective amount of a zileuton-containing composition and an effective amount of a nephrotoxicity-inducing anti-cancer drug. The anti-cancer drug can be one or more of ifosfamide, ipilimumab, pembrolizumab, and nivolumab. In certain examples, the effective amount of zileuton can range from about 0.5 mg/kg to about 200 mg/kg.

In certain examples, the zileuton-containing composition can be administered intravenously, intraperitoneally, subcutaneously, intramuscularly, intranasally, intrathecally, or transdermally to a subject.

Embodiments further include kits for parenteral administration of zileuton. In certain examples, the kits include instructions for use and a pharmaceutical composition containing a therapeutically effective amount of zileuton and a biologically acceptable excipient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to illustrate embodiments of the disclosure more clearly.

FIGS. 1A-1B depict chromatograms of zileuton (the USP standard). FIG. 1A shows the separation of analytes by reverse-phase liquid chromatography and detection based on retention times and mass/charge ratios, respectively. Detection was performed using the multiple reaction monitoring (MRM) scan type in the positive-ion mode. Zileuton-d4 was used as the internal standard (shown in blue on the chromatogram). In FIG. 1B, the linear range of the assay was 0.0625-8 μg/mL in rat serum (r²=0.997).

FIG. 2 is a graphical representation of the solubility of zileuton in various solvents generally regarded as safe (GRAS) by the FDA for parenteral pharmaceutical formulation.

FIG. 3 depicts a ternary diagram of solubility for zileuton (1 mg/mL). Data for an example formulation according to an embodiment is shown by a star (65% water, 25% PEG 400, 10% ethanol).

FIG. 4 is a graphical representation of the stability of zileuton in an example formulation (65% water, 25% PEG 400, 10% ethanol).

FIGS. 5A-5D are graphical representations of the pharmacokinetic profiles of zileuton in serum (FIG. 5A: 4 mg/kg, FIG. 5C: 12 mg/kg) and renal tissue (FIG. 5B: 4 mg/kg, FIG. 5D: 12 mg/kg) in rats.

FIG. 6 is a graphical representation of the pharmacokinetics of zileuton at steady state or following single-dose administration. To provide steady state, zileuton (12 mg/kg) in an example formulation (65% water, 25% PEG 400, 10% ethanol) was given once daily for 10 days.

FIG. 7 is a graphical representation of the onset of nephrotoxicity associated with amikacin in rats. Animals in both cohorts received amikacin (AMK) 300 mg/kg daily. Both cohorts (administered or not administered zileuton) comprised 10 animals (5 males and 5 females). The endpoint was defined as ≥2× elevation of serum creatinine value from baseline for each animal. p=0.015.

FIGS. 8A-8C depict histological evidence of renal injury in kidney tissue of rats administered amikacin (FIG. 8A), vehicle (FIG. 8B), or amikacin and zileuton (FIG. 8C). The histology sections were PAS stained and shown in 400×.

FIG. 9 is a graphical representation of the onset of nephrotoxicity associated with polymyxin B in rats. Animals in both cohorts received polymyxin B (PB) 20 mg/kg daily. Both cohorts (first cohort administered PB alone, and second cohort administered PB and zileuton) comprised 10 animals (both genders). The polymyxin B alone group consisted of 5 animals receiving the vehicle and 5 animals that received no vehicle. The two control groups were deemed comparable (thus combined for the analysis) as the median time to reach nephrotoxicity was 5 versus 6 days, respectively. The endpoint was defined as ≥2× elevation of serum creatinine value from baseline for each animal. p<0.01.

FIGS. 10A and 10B are graphical representations of the dose-response (reduction in nephrotoxicity) associated with zileuton. FIG. 10A depicts the reduction in nephrotoxicity associated with 300 mg/kg amikacin with concomitant zileuton (1 and 4 mg/kg). FIG. 10B depicts reduction in nephrotoxicity associated with 20 mg/kg polymyxin B with concomitant zileuton (4 and 10 mg/kg). Nephrotoxicity was defined as a ≥2× elevation of each animal's baseline serum creatinine over 10 days. N=10 in each group.

FIGS. 11A-11C are graphical representations of the multi-dose safety of zileuton. Zileuton 12 mg/kg in an example formulation (in 65% water, 25% PEG 400, 10% ethanol) was administered to rats once daily for 10 days (day 1-10), and weight (FIG. 11A), serum creatinine (FIG. 11B), and ALT (FIG. 11C) were measured at baseline, day 5, day 10, or day 14 as indicated.

FIG. 12 schematically depicts nanoparticles (NPs) and the dynamic light scatter analysis of NPs showing a monodisperse peak with mean diameter of 280 nm and a zeta potential of −3 mV.

FIGS. 13A-13C depict in vivo imaging of near-infrared dye conjugated AF750-NPs in nude mice. FIG. 13A shows fluorescence imaging of NPs in mice for up to 72 hrs. FIG. 13B shows ex vivo fluorescence imaging of major organs 3 days post IV injection of NPs. LIV: liver, SPL: spleen, NK: normal kidney. FIG. 13C shows multispectral optoacoustic tomography (MOST) imaging of mouse kidneys 3 days after IV AF750-NPs administration. The green color indicates the fluorescent NP. N=3 mice per imaging group.

FIG. 14A-14D depict fluorescence images (40×) of the kidney after 3 days post IV administration of AF750-labeled NPs in mice. Kidneys were stained with anti-mouse megalin antibody and cell nuclei were stained with DAPI (Red: NPs (FIG. 14A), Green: megalin (FIG. 14B), Blue: nuclei (FIG. 14C), and all overlaid in FIG. 14D) (n=3). Specific targeting of the proximal tubular cells of kidney in mice were evaluated.

FIGS. 15A-15B depict an expanded solubility screen of zileuton in ethanol:PEG 400:water blends suitable for a parenteral formulation. FIG. 15A provides a graphical visualization of the ethanol, PEG 400, and water ratios for the zileuton formulations. FIG. 15B shows a tabulated matrix of formulations A-V listing component ratios and the corresponding measured solubility of zileuton (mg/mL).

FIGS. 16A-16C are graphical representations of the long-term (shelf) stability of zileuton formulations: 10 mg/mL zileuton in ethanol (FIGS. 16A), 1 mg/mL zileuton in 65% water, 25% PEG 400, 10% ethanol (FIG. 16B), and 10 mg/mL zileuton in 25% water, 50% PEG 400, 25% ethanol (FIG. 16C).

FIGS. 17A-17C depict tolerance and safety data of the zileuton formulations when administered to rats at 12 mg/kg zileuton daily intraperitoneally. Weight (FIG. 17A), ALT (FIG. 17B), and serum creatinine (FIG. 17C) were measured at baseline, day 10, and day 14 post-administration.

FIGS. 18A-18B summarize exposure and relative bioavailability of zileuton across administration routes at a 4 mg/kg dose. FIG. 18A presents zileuton plasma concentration over time following IV, IP, and PO administration. FIG. 18B shows mean area under the concentration-time curve (AUC) values and the corresponding bioavailability estimates for IP, PO, and IV administration.

FIG. 19 is a tabular projection relating rat intraperitoneal doses of zileuton to putative human oral and intravenous doses expected to yield comparable systemic exposure, based on cross-route AUC relationships. The table anchors the projections to the known clinical daily oral dose of 2,400 mg and provides illustrative matched human IV dose estimates under the stated assumptions.

FIGS. 20A-20B are graphical representations of serum creatinine values following bilateral renal ischemia-reperfusion injury in female Sprague-Dawley rats. Animals (225-250 g; n=6 per group) underwent 30 minutes of bilateral renal artery clamping under isoflurane anesthesia with temperature maintained at 38±1° C., and were then randomized to normal saline (FIG. 20A) or zileuton (FIG. 20B) treatment. Zileuton was administered intraperitoneally at 6 mg/kg for three doses given 10 hours and 2 hours prior to surgery and 6 hours after surgery. Blood was collected at baseline, and at 1, 2, and 5 days post-surgery; serum creatinine was measured using a Piccolo Xpress Chemistry Analyzer. The acute kidney injury endpoint was defined as a ≥2-fold increase from each animal's baseline serum creatinine.

FIGS. 21A-21B are graphical representations of blood urea nitrogen (BUN) trajectories in the same bilateral renal ischemia-reperfusion model in female Sprague-Dawley rats, comparing normal saline controls (FIG. 21A) with zileuton-treated animals (FIG. 21B). Animals (n=6 per group) received the dosing regimen described for FIGS. 20A-20B, and BUN was measured at baseline and at 1, 2, and 5 days post-surgery using a Piccolo Xpress Chemistry Analyzer to assess renal functional recovery over time.

### DETAILED DESCRIPTION

The description may use the phrases “in certain examples,” “in certain embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The term “about” or “approximately” is defined as being close to as understood by one of ordinary skill in the art. In one non-limiting embodiment, the terms are defined to be within 10%, preferably within 5%, more preferably within 1%, and most preferably within 0.5%. The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The term “plurality” as used herein refers to two or more items or components. The terms “wt. %”, “vol. %”, and “mol. %” refer to a weight, volume, or molar percentage of a component, respectively, based on the total weight, the total volume of material, or total moles, that includes the component. In a non-limiting example, 10 grams of component in 100 grams of the material is 10 wt. % of component.

Zileuton contains one weakly acidic hydrogen atom and one asymmetric center and thus gives rise to the formation of salt forms and enantiomers that may be defined, in terms of absolute stereochemistry, as (R)- or(S)- and in terms of their optical rotations as (+) and (−) enantiomers. The present disclosure includes all salt forms and racemic mixtures, optically pure forms, and cyclodextrin derived inclusion complex mixtures. The sodium salt of zileuton is commercially available, and the optically active (R)- and (S)-isomers may be prepared using known chiral synthons, chiral reagents, or separated into pure enantiomers using other means known in the art. “Stereoisomers” are isomers that differ only in the way the atoms are arranged in space. “Enantiomers” are a pair of stereoisomers that are non-superimposable mirror images of each other. A 1:1 mixture of a pair of enantiomers is called a “racemic” mixture. The term “(±)” is used to designate a racemic mixture where appropriate. The absolute stereochemistry is specified according to the Cahn-Ingold-Prelog R-S system. When a compound is a pure enantiomer, the stereochemistry at each chiral carbon may be specified by either R or S. Resolved compounds whose absolute configuration is unknown can be designated (+) or (−) depending on the direction (dextro-or levorotatory) in which they rotate plane polarized light at the wavelength of the sodium D line. It is intended that the compounds described herein include racemates, both E and Z geometric isomers, and various pharmaceutically acceptable salt forms. As used, herein, the term “zileuton” encompasses ((±) l-[1-(l-benzothiophen-2-yl)ethyl]-1-hydroxyurea, the optically pure form of the (S)-enantiomer or (−)-isomer of N-(l-benzo[b]thien-2-ylethyl)-N-hydroxyurea (as described, for example, in U.S. Pat. No. 5,629,337), the optically pure form of (R)-enantiomer or (+)-isomer of N-(1-benzo[b]thien-2-ylethyl)-N-hydroxyurea (as described, for example, in WO 94/26268) and mixtures of the (S)- and (R)-isomers in any ratio between 1:99 and 99:1, and polymorphic forms of zileuton. Certain embodiments include the zileuton salts, such as the sodium salt of zileuton (sodium; l-[1-(lbenzothiophen-2-yl)ethyl]-1-oxidourea).

As used herein, a “subject” is an animal, such as a mammal, including a primate (such as a human, a non-human primate, for example a monkey) and a non-primate (such as a cow, a dog, a horse, a sheep, a rabbit, a cat, a rat, or a mouse). In some embodiments, the subject is a human. This subject can be a human having, suspected of having, or at risk of developing, infection or cancer for which treatment with a nephrotoxicity-inducing drug is indicated. The subject can be an adult subject or a pediatric subject, such as a neonate, an infant, or a child.

As used herein, “treating” a condition or “treatment” of a condition (for example nephrotoxicity, renal insufficiency, or AKI associated with use of a nephrotoxicity-inducing agent for, for example treatment of infection or cancer) refers to a beneficial or desired result, such as reducing at least one sign, symptom, or complication associated with the condition. “Treating” or “treatment” also refers to delaying the onset or progression of the condition, or signs, symptoms, or complications associated with the condition in a subject. “Treating” or “treatment” also refers to a preventive or prophylactic treatment, such as prevention of a condition or prevention of at least one sign, symptom, or complication associated with the condition. Accordingly, “treatment” can refer to a reduction in likelihood of developing a disease or associated signs, symptoms, conditions, or complications, or a reduction in severity of a disease or associated signs, symptoms, conditions, or complications relative to a population having the same risk factors and not receiving treatment as described herein. The failure to develop a disease, or a delay in time to develop associated signs, symptoms, conditions, or complications by days, weeks, months, or years is considered effective treatment. Treatment may require administration of more than one dose of the pharmaceutical compositions described elsewhere herein. “Treatment” can also mean prolonging survival as compared to expected survival in the absence of treatment.

The term “reducing,” “reduced,” “decreasing,” “decreased,” or any variation thereof, when used in the claims and/or the specification includes any measurable decrease of one or more components in a mixture to achieve a desired result, such as a detectable (for example at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter from a comparison control, for example an established normal or reference level of the parameter, or an established standard control. Accordingly, the terms “reduced,” “decreased,” and the like encompass both a partial reduction and a complete reduction compared to a control.

The term “increased,” “increasing,” “increase,” or any variation thereof refers to a detectable (for example at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100%, 120%, 150%, 200%, 300%, 400%, 500%, or more) positive change in the parameter from a comparison control, for example an established normal or reference level of the parameter, or an established standard control. Accordingly, the terms “increased” and the like encompass both a partial increase and a significant increase compared to a control.

Zileuton is a commercially available 5-lipoxygenase inhibitor indicated for the treatment of chronic asthma. Compositions and methods containing zileuton for treating diseases or conditions other than asthma are described in WO2020/092016.

Certain embodiments of this disclosure include parenteral compositions for treating bacterial infections or cancer in a mammal containing an effective amount of zileuton or a pharmaceutically acceptable salt thereof and an effective amount of one or more nephrotoxicity-or neurotoxicity-inducing antibiotics or anti-cancer drugs, respectively, and methods of treating infections or cancer in a mammal by administering to the mammal an effective amount of the parenteral composition. Also provided herein are parenteral compositions for treating acute kidney injury or diabetic nephropathy with an effective amount of zileuton or a pharmaceutically acceptable salt thereof, and methods of treating acute kidney injury or diabetic nephropathy in a mammal by parenterally administering to the mammal an effective amount of the composition.

Globally, the prevalence of antimicrobial resistance (AMR) has been increasing for decades. Resistance to commonly used antibiotics (such as beta-lactams and fluoroquinolones) is widespread and presents an ongoing public health concern. Antibiotics used for difficult-to-treat infections (such as aminoglycosides and polymyxins) have maintained in vitro activity against multidrug-resistant (MDR) Gram-negative bacteria such as A. baumannii, K. pneumoniae, and Pseudomonas aeruginosa, which have been identified by the Infectious Diseases Society of America as warranting urgent attention. However, aminoglycosides and polymyxins are associated with AKI, which can limit their clinical use to treat MDR Gram-negative bacterial infections. The rise of AMR has increased interest in identifying strategies to reduce the renal toxicity of these antibiotics. Due to the low prevalence of resistance in most bacteria and the widespread availability of these antibiotics, attenuation of amikacin-and polymyxin B-associated AKI would be a significant and timely accomplishment to combat the AMR crisis. As disclosed herein, Applicant has developed the use of zileuton to reduce aminoglycoside-and polymyxin-associated nephrotoxicity associated with AKI.

The zileuton composition for parenteral administration provided herein can be prepared by combining a therapeutically effective amount of zileuton with a biologically acceptable excipient (for example solvent or nanoparticle). The zileuton composition can contain at least about 1 mg/ml of zileuton.

Provided herein are methods of administering the zileuton composition parenterally to a subject, such as to treat or prevent a disease or a condition. For example, the pharmaceutical composition can be used in preventing, delaying the onset of, or treating AKI or nephrotoxicity in a subject. In certain examples, the AKI or nephrotoxicity is induced by an antibiotic, a diagnostic contrast agent, or an anti-cancer drug such as those provided herein, or is associated with ischemia-reperfusion injury related to surgery, shock, or organ transplantation.

With respect to the compositions and methods for treating bacterial infections, the nephrotoxicity- or neurotoxicity-inducing antibiotics can be one or more of amikacin, tobramycin, netilmicin, streptomycin, arbekacin, plazomicin, neomycin, kanamycin, paromomycin, gentamicin, bacitracin, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815, and octapeptin C4. The aforementioned composition can further include one or more of amikacin, apramycin, azithromycin, aztreonam, menopenem-vaborbactam, imipenem-relebactam, ceftazidime-avibactam, ceftolozane-tazobactam, chloramphenicol, clindamycin, daptomycin, doxycycline, eravacycline, erythromycin, fosfomycin, fusidic acid, levofloxacin, linezolid, Lpxc inhibitor CHIR-090, meropenem, minocycline, rifampin, spectinomycin, tetracycline, tigecycline, trimethoprim-sulfamethoxazole, vancomycin, and gentamicin.

With respect to the compositions and methods for treating cancer, the nephrotoxicity- or neurotoxicity-inducing anti-cancer drugs can be one or more of ifosfamide or an immune checkpoint inhibitor such as, for example, ipilimumab, pembrolizumab, or nivolumab; and a pharmaceutically acceptable carrier. In certain examples, the nephrotoxicity-inducing anti-cancer drugs include platinum-based chemotherapeutic agents such as cisplatin and carboplatin, antimetabolites such as methotrexate, and alkylating agents such as ifosfamide.

Also provided are methods of treating infection, inflammation, cancer, acute kidney injury, and/or diabetic nephropathy in a subject by administering zileuton or a composition containing zileuton to the subject, and suitable formulation and dosage thereof, including extended use. “Extended use” of zileuton as used herein refers to therapeutic use of a zileuton composition beyond what is originally approved by the FDA, which is for treating or preventing asthma. Extended use of zileuton can be for any disease or condition for which use of zileuton is therapeutically beneficial, including for preventing, delaying the onset of, or treating nephrotoxicity or AKI associated with use of a nephrotoxicity-inducing agents for infection or cancer treatment. Extended use includes parenteral administration, intravenous administration, intraperitoneal administration, subcutaneous administration, intramuscular administration, transdermal administration, continuous use, and non-continuous use (for example periodic, regular, or repeated use) for the duration of time in which the zileuton composition was used (for example administered to the subject). Extended use further includes prophylactic perioperative administration to prevent ischemia-reperfusion injury in settings such as cardiac surgery, vascular surgery, organ transplantation, and procedures involving a diagnostic contrast agent.

In one aspect, provided herein is a method of treating a subject with infection, by administering parenterally an effective amount of the zileuton composition and an effective amount of a nephrotoxicity-inducing antibiotic selected from the group consisting of amikacin, tobramycin, netilmicin, streptomycin, arbekacin, plazomicin, neomycin, kanamycin, paromomycin, gentamicin, bacitracin, polymyxin B, colistin, amphotericin B, tetracyclines, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815, and octapeptin C4.

In one aspect, provided herein is a method of treating a subject with cancer, by administering parenterally an effective amount of the zileuton composition provided herein and an effective amount of a nephrotoxicity-inducing anti-cancer drug, such as ifosfamide, ipilimumab, pembrolizumab, and nivolumab. In certain examples, the nephrotoxicity-inducing anti-cancer drug is cisplatin, carboplatin, or methotrexate.

In certain examples, the effective amount of zileuton is about 0.5-200 mg/kg. Zileuton can be administered by any route suited for clinical purposes, for example, intravenously, intraperitoneally, subcutaneously, intramuscularly, or transdermally. The zileuton composition can be administered in a single dose or for multiple doses for an extended time period.

Currently, zileuton is formulated as tablets for oral administration. These tablets may be regular, film-coated, or extended release forms. However, in the target subject population for extended use of zileuton (for example, critically ill subjects, subjects with hemodynamic instability, and/or subjects with multidrug-resistant bacterial infections for whom polymyxins are used as last resort treatment), the oral route may be less reliable for systemic delivery of the active ingredient to achieve a therapeutic effect. Accordingly, provided herein is a pharmaceutical composition formulated for a parenteral route of drug administration (for example, as an intravenous infusion). Also provided herein is a method of preparing the pharmaceutical composition. The parenteral zileuton composition can be prepared by combining a therapeutically effective amount of zileuton with a biologically acceptable excipient.

In some aspects, provided herein is a co-solvent system for parenteral administration of zileuton. Zileuton (MW=236) has a log P value of 0.9 and is practically insoluble in water (<0.5 mg/mL). In certain examples, the co-solvent parenteral formulation for zileuton contains FDA-approved excipients. In certain examples, the excipient is polyethylene glycol (PEG). The PEG can range from about 20 vol. % to about 50 vol. % of the excipient. The excipient component can include PEG with a molecular weight ranging from about 350 to about 650 Daltons (such as PEG 400 , PEG 500, PEG 600, or PEG 400-600), propylene glycol, glycerin, ethanol, sorbitol, or any combinations thereof. In certain examples, the excipient contains ethanol, PEG 400, and water in 1:4:5 or 10%:40%:50% by volume. In certain examples, the excipient contains about 10 vol. % ethanol, about 40 vol. % PEG 400, and about 50 vol. % water. In certain examples, the excipient contains ethanol, PEG 400, and water in 10%:25%:65% by volume. In certain examples, the excipient contains about 10 vol. % ethanol, about 25 vol. % PEG 400, and about 65 vol. % water. The formulation can generate a 1-10 mg/mL zileuton injectable solution to support preclinical or clinical investigations. The target dose of zileuton can be determined based on various clinical factors (for example, subject weight). The injection volume can be adjusted accordingly. For instance, in the rodent model, 1 mg/mL zileuton with a volume ranging from 1 mL to 3 mL can be given intraperitoneally.

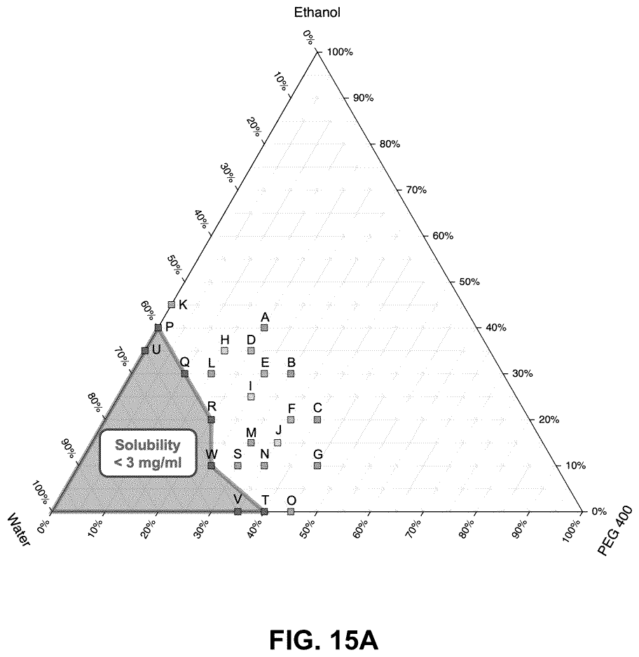

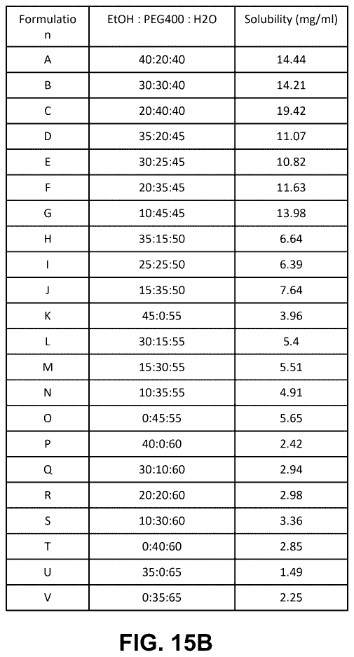

As shown in FIGS. 15A-15B, an expanded ethanol:PEG 400:water composition matrix can be used to select formulations that maximize zileuton solubility while remaining suitable for parenteral administration. Formulation A-V in FIG. 15B list component ratios and the corresponding measured solubility of zileuton (mg/mL), and FIG. 15A provides graphical visualization of these data to delineate composition regions that maximize solubility within the ethanol/PEG 400/water system. Final compositions can be chosen from within solvent regions that satisfy both solubility and safety constraints.

Zileuton can first be dissolved in ethanol as a solution of 10 mg/ml and stored at −20° C. Immediately prior to dosing, 0.1 ml of the ethanol solution can be diluted 1:9 with PEG 400:water (4:5 co-solvent) to provide a 1 mg/ml final concentration of zileuton. Based on the target dose (for example based on subject weight), the injection volume can be adjusted accordingly when given intraperitoneally or via any suitable parenteral route, including but not limited to intraperitoneally, subcutaneously, intramuscularly, or transdermally. Zileuton can also be prepared by dissolving in a solvent containing ethanol, PEG 400, and water in a ratio of 10%:25%:65%.

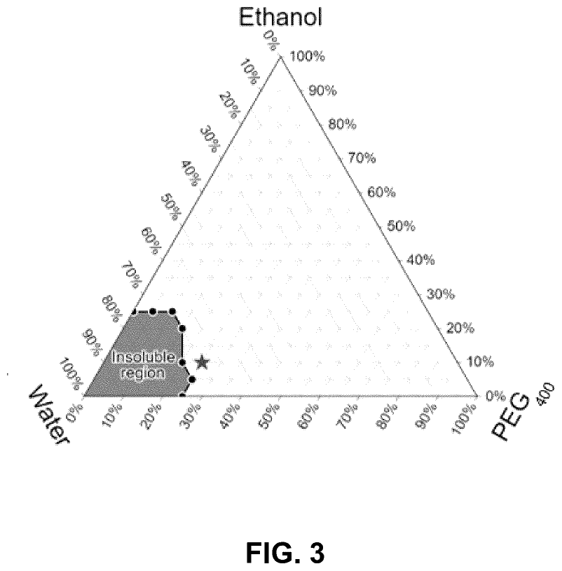

In certain examples, an aqueous composition is provided for parenteral administration containing a therapeutically effective amount of zileuton and a biologically acceptable excipient, for renal protection against a nephrotoxicity-inducing agent. In certain examples, the excipient is polyethylene glycol, PEG 400-600, propylene glycol, glycerin, ethanol, sorbitol, triacetin, polyoxyethylated glycerides, polyoxyethylated oleic glycerides, hydroxypropyl-beta-cyclodextrin, a surfactant (e.g. hydroxypropylcellulose 20, polysorbate 80, polysorbate 20, sorbitan monooleate NF, polyoxyl 40 hydrogenated castor oil, polyoxyl hydroxystearates), or combinations of any thereof. Any combination of the excipients in any ratio can be used, optionally in combination with water. For example, in certain examples, the excipient is ethanol, PEG 400, and water in 1:4:5 or 10%:25%:65% by volume or in any combination presented by the ratio of ethanol, PEG 400, and water that is outside the insoluble region of FIG. 3 and/or within composition ranges identified for solubility in FIGS. 15A-15B.

“Therapeutically effective amount” or “effective amount” refers to that amount of an agent (for example zileuton) effective to produce the intended pharmacological, therapeutic, or preventive result, such as renal protective effect against nephrotoxicity-inducing agents. An effective amount also may include an amount effective to inhibit or reduce signs or symptoms associated with nephrotoxicity (for example decrease in urine output, decrease in creatinine clearance). For example, if a given clinical treatment is considered effective when there is at least a 10% improvement (for example increase or decrease that relates to improvement of the disease, condition, or associated signs or symptoms) in a measurable parameter associated with a disease or disorder, a therapeutically effective amount of a drug for the treatment of that disease or disorder is the amount necessary to obtain at least a 10% improvement (for example by at least 10%, 15%, 20%, 25%, 30%, 35%, 40%, 45%, 50%, 55%, 60%, 65%, 70%, 75%, 80%, 85%, 90%, or 95%) in that parameter.

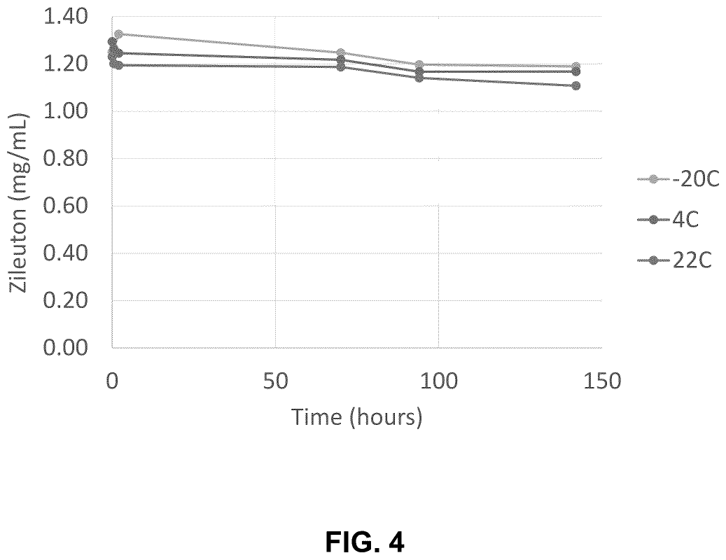

The zileuton formulation can be stable. For example, the parenteral zileuton composition degrades less than 10% over 140 hours to 150 hours at −20° C., 4° C., or 22° C. As shown in FIG. 4, an example formulation containing 65% water, 25% PEG 400, and 10% ethanol exhibited no significant degradation over at least 142 hours under these storage conditions.

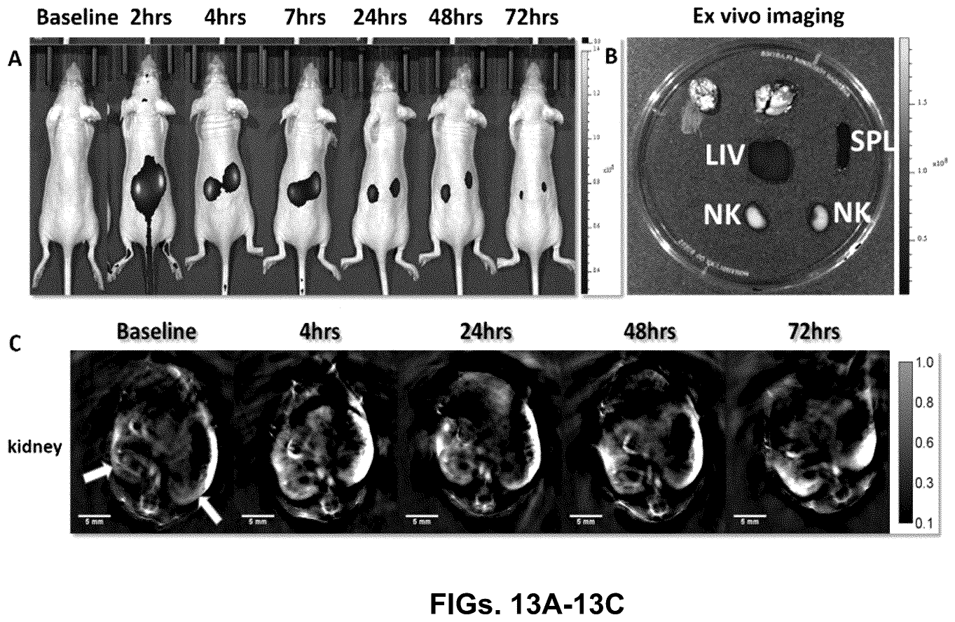

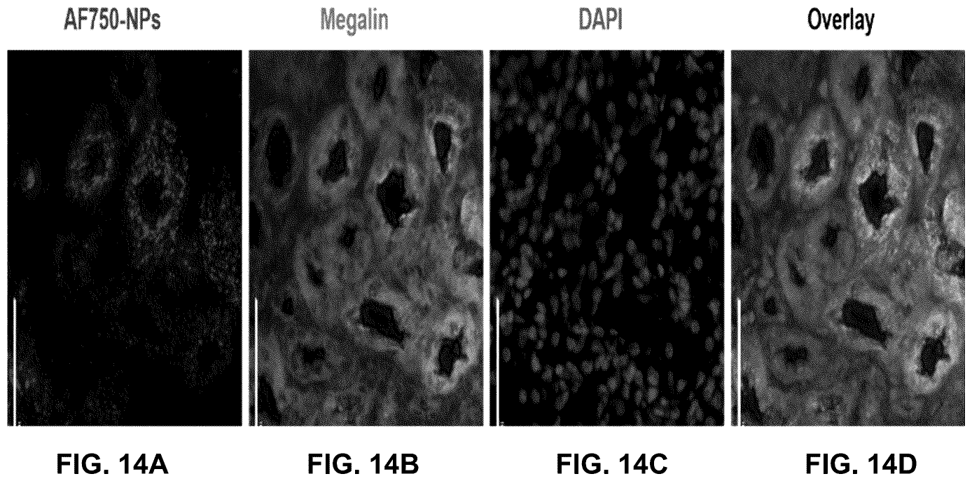

In some aspects, provided herein is a stable formulation of zileuton for parenteral administration. The stable parenteral formulation of zileuton can include biocompatible nanoparticle compositions that are selectively taken up by renal proximal tubular cells, resulting in an enhanced uptake of renal protectant at the site of renal injury. As shown in FIGS. 13A-13C and 14A-14D, fluorescently labeled nanoparticles accumulated in the kidney and localized to megalin-positive proximal tubular epithelium without observable toxicity, supporting selective renal targeting.

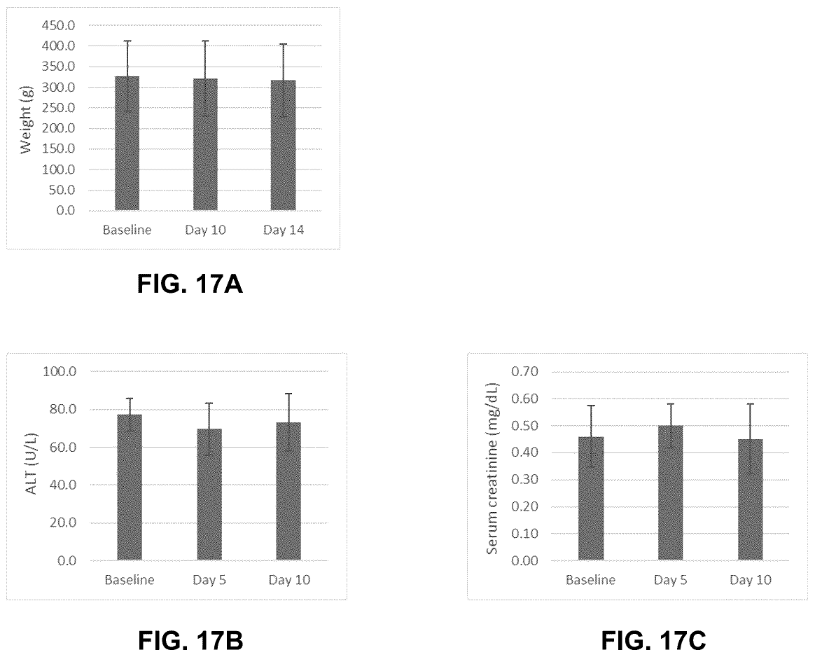

The tolerance and safety of zileuton formulations for repeated parenteral administration have been evaluated. As shown in FIGS. 17A-17C, administration of 12 mg/kg zileuton daily intraperitoneally to rats was well tolerated, with no significant changes in body weight (FIG. 17A), serum ALT (a marker of hepatocellular toxicity) (FIG. 17B), or serum creatinine (a marker of renal function) (FIG. 17C) measured at baseline, day 10, and day 14 post-administration. These data support the safety of repeated parenteral zileuton administration at therapeutically relevant doses.

In certain embodiments, nanoparticles composed of polysaccharide, pegylated lipids, and phosphate counter ions exhibit a mean hydrodynamic diameter of about 280 nm and a near-neutral zeta potential of about −3 mV. Following intravenous administration, in vivo and ex vivo imaging demonstrated sustained kidney accumulation over multiple days, with fluorescence colocalizing to megalin-positive proximal tubular cells, consistent with endocytic uptake by proximal tubule epithelium. Nanoparticles are ultrafine particles that generally have at least one dimension of less than 100 nm in size. Nanoparticles made of biocompatible and biodegradable materials can offer many pharmacokinetic, efficacy, and safety benefits when they are used as drug formulations. Various therapeutic and diagnostic agents can be encapsulated in nanoparticles for targeted delivery. The property and in vivo performance of nanoparticles as drug delivery vehicles are crucially dependent on the nature of biomaterial used, size, surface charge, rigidity, permeability, solubility, stability, etc.

There are two ways through which nanostructures deliver drugs: passive and self-delivery, both of which are within the scope of the present disclosure. In the former, drugs are incorporated in the inner cavity of the structure mainly via the hydrophobic effect. When the nanostructure materials are targeted to specific sites, the intended amount of the drug is released because of the low content of the drugs which is encapsulated in a hydrophobic environment. Conversely, in the latter, the drugs intended for release are directly conjugated to the carrier nanostructure material for easy delivery. In this approach, the timing of release is crucial as the drug will not reach the target site if it dissociates from the carrier very quickly, and conversely, its bioactivity and efficacy will be decreased if it is released from its nanocarrier very slowly. Targeting of drugs is another significant aspect that uses nanomaterials or nanoformulations as the drug delivery systems and is classified into active and passive. In active targeting, moieties, such as antibodies and peptides, are coupled with drug delivery system to anchor them to the receptor structures expressed at the target site. In passive targeting, the prepared drug carrier complex circulates through the bloodstream and is driven to the target site by affinity or binding influenced by properties like pH, temperature, molecular site, and shape. The main targets in the body are the receptors on cell membranes, lipid components of the cell membrane, and antigens or proteins on the cell surfaces.

In one aspect, provided herein is a nanoparticle-based composition containing a therapeutically effective amount of zileuton and a nanoparticle, formulated for parenteral administration for, such as renal protection against a nephrotoxicity-inducing agent. The nanoparticle contains polysaccharide, a pegylated lipid, and/or phosphate counter ions.

The size of the nanoparticle can be large enough not to easily cross the glomerular filtration barriers and be excreted in the urine (for example ≥6 nm), and not too big to be cleared by organs of the reticuloendothelial system (RES) (for example ≤500 nm). In certain examples, the nanoparticle contains a mean hydrodynamic diameter of about 50-300 nm (for example about 50 nm, 60 nm, 70 nm, 80 nm, 90 nm, 100 nm, 150 nm, 200 nm, 250 nm, 300 nm, 50-100 nm, 100-150 nm, 150-200 nm, 200-250 nm, 250-300 nm) and a near neutral charge. In certain examples, the nanoparticle contains a mean hydrodynamic diameter of about 280 nm and a mean zeta potential of about −3 mV. In certain examples, the composition accumulates in proximal tubule epithelia when administered to subjects. In certain examples, zileuton is directly or externally conjugated to nanoparticles, which can deliver zileuton to the target tissue, for example proximal tubule epithelia of the kidney (for example active targeting). In certain examples, zileuton is incorporated in the inner cavity of nanoparticles, which can deliver zileuton to the target tissue, for example proximal tubule epithelia of the kidney (for example passive targeting).

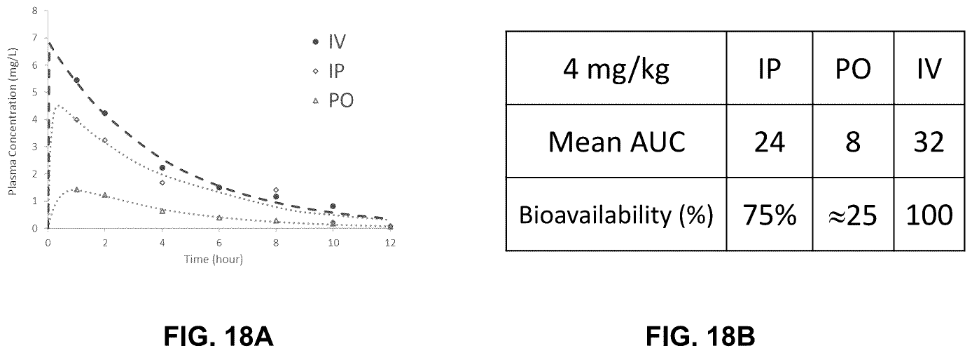

Pharmacokinetic considerations support parenteral administration of zileuton to achieve reliable systemic exposure. As shown in FIGS. 18A-18B, mean area under the concentration-time curve (AUC) values for intraperitoneal (IP), oral (PO), and intravenous (IV) dosing at 4 mg/kg were 24, 8, and 32 μg·h/mL, respectively, and relative bioavailability with intraperitoneal administration (75%) approaches that of intravenous administration (100%) and substantially exceeds oral bioavailability (approximately 25%). These data support selection of parenteral routes for prophylaxis or treatment of AKI.

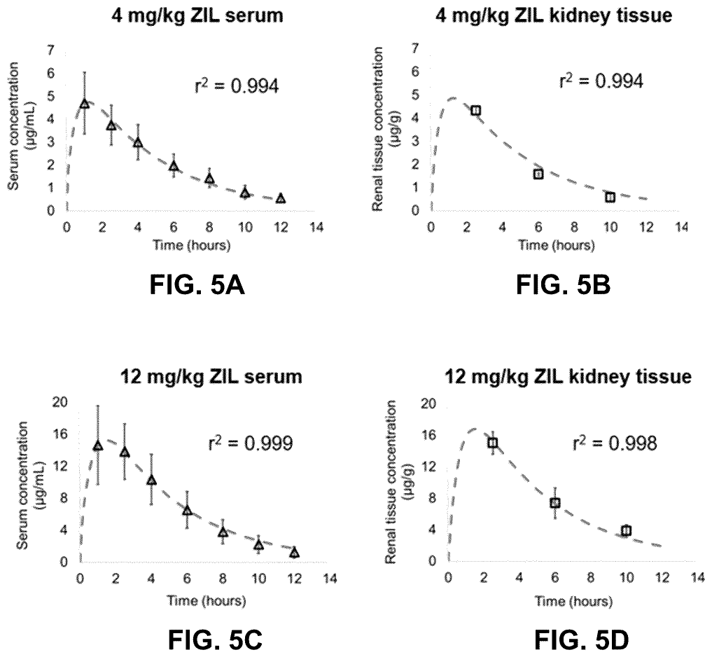

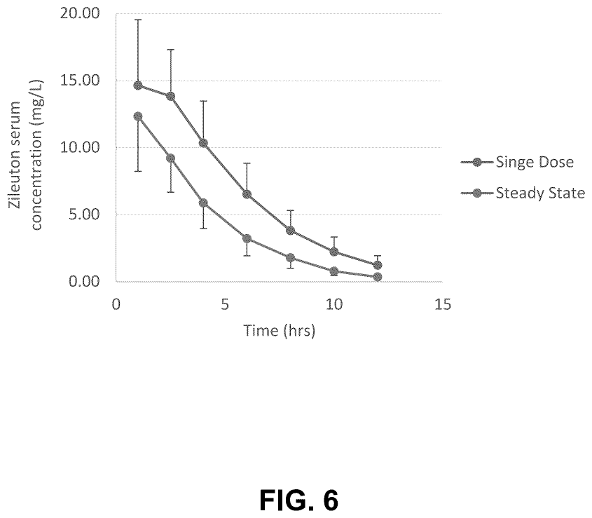

A subject (for example a human subject, for example a patient) can be administered a therapeutic amount of the zileuton composition. The therapeutic amount can be, for example, 0.05-50 mg/kg body weight. In certain examples, the therapeutic amount of zileuton delivered as a parenteral formulation is from about 1 mg/kg to 12 mg/kg, for example about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, or about 12 mg/kg. In certain examples, a single parenteral dose of 4 mg/kg in rats corresponds by proportional exposure to a human oral dose equivalent of approximately 880 mg, and a 12 mg/kg rat dose corresponds to approximately 3000 mg, supporting dose selection and clinical translation. See FIGS. 5A-5D and 6 for representative exposure profiles supporting these equivalencies.

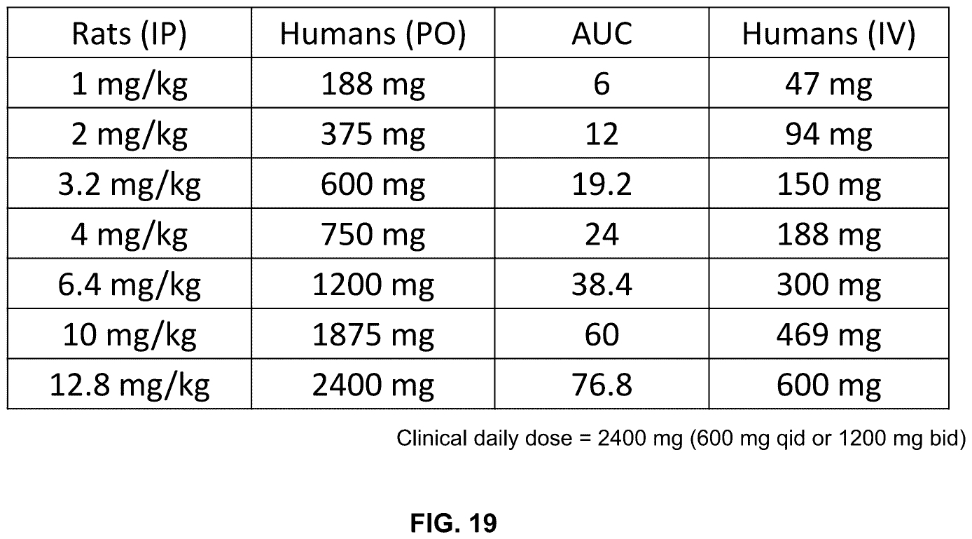

Dose selection for human administration can be informed by cross-route exposure relationships and clinical dose anchoring. As shown in FIG. 19, rat parenteral doses can be related to putative human oral and intravenous doses expected to yield comparable systemic exposure, anchored to a known clinical daily oral dose of 2,400 mg (ZYFLO®). For example, rat intraperitoneal doses of 4 mg/kg and 12 mg/kg correspond to projected human intravenous doses that can be calculated based on proportional AUC, thereby supporting initial clinical dosing strategies for parenteral formulations.

The zileuton composition of the present disclosure can be administered to a subject via any methods or routes that are suited for the subject. For example, the zileuton composition can be administered via parenteral routes, including intracranial (for example intraventricular, intraparenchymal, and intrathecal), intraperitoneal, intravenous (for example injection, infusion), intramuscular, subcutaneous, transdermal, airway (aerosol), nasal, ocular, and topical (including buccal and sublingual) administration. Zileuton compositions can be formulated for delivery to a target organ, for example to the kidney. In certain examples, the zileuton composition is administered to promote deposition substantially in the kidney, such as the proximal tubule.

In certain embodiments, the zileuton composition is taken up in one or more tissues or cell types in the renal system including, but not limited to, tubular epithelia, macula densa, glomerular endothelia, podocytes, mesangial cells, glomerular basement membrane cells, and parietal epithelia. In specific embodiments, the zileuton composition accumulates in tubular epithelia of the kidney, for example proximal tubular epithelia.

In certain examples, the administration is repeated, for example, on a regular basis, such as, daily, biweekly (i.e., every two weeks) for one month, two months, three months, four months, six months or longer. After an initial treatment regimen, the treatments can be administered on a less frequent basis. For example, after administration bi-weekly for three months, administration can be repeated once per month, for six months, or a year, or longer. In perioperative or procedure-related settings, zileuton can be administered on a short-course prophylactic schedule, for example, as multiple doses bracketing the anticipated ischemic insult or nephrotoxic exposure.

In certain examples, the parenteral zileuton composition is administered in two or more doses. In certain examples, the number or amount of subsequent doses is dependent on the achievement of a desired effect, for example to inhibit or reduce kidney damage associated with co-administration of nephrotoxicity-inducing agents, for example reduction or prevention of one or more symptoms associated with acute kidney injury.

In certain examples, the parenteral zileuton composition is administered according to a schedule. For example, the parenteral zileuton composition may be administered once per week, twice per week, three times per week, four times per week, or five times per week. In certain examples, the schedule involves regularly spaced administrations, for example hourly, every four hours, every six hours, every eight hours, every twelve hours, daily, every 2 days, every 3 days, every 4 days, every 5 days, weekly, biweekly, or monthly. In certain examples, the parenteral zileuton composition is administered at the frequency required to achieve a desired effect. In certain perioperative embodiments relevant to ischemia-reperfusion injury, zileuton is administered as three doses given approximately 10 hours and 2 hours prior to the ischemic event and 6 hours after reperfusion.

In certain examples, the schedule involves closely spaced administrations followed by a longer period of time during which the agent is not administered. For example, the schedule may involve an initial set of doses that are administered in a relatively short period of time (for example about every 6 hours, about every 12 hours, about every 24 hours, about every 48 hours, or about every 72 hours) followed by a longer time period (for example about 1 week, about 2 weeks, about 3 weeks, about 4 weeks, about 5 weeks, about 6 weeks, about 7 weeks, or about 8 weeks) during which the parenteral zileuton composition is not administered. In certain examples, the parenteral zileuton composition is initially administered hourly and is later administered at a longer interval (for example daily, weekly, biweekly, or monthly). In certain examples, the parenteral zileuton composition is initially administered daily and is later administered at a longer interval (for example weekly, biweekly, or monthly). In certain embodiments, the longer interval increases over time or is determined based on the achievement of a desired effect. In the course of zileuton administration, different routes of administration can be employed (for example intravenous administration in the initial administration, followed by subcutaneous administration in the second administration). In procedure-related embodiments, administration can be confined to the peri-procedural window and discontinued upon stabilization of renal function.

Before administration of a full dose of the zileuton composition, subjects can be administered a smaller dose, such as a 5% infusion dose, and monitored for adverse effects, such as an allergic reaction, or for elevated lipid levels or blood pressure. In another example, the subject can be monitored for unwanted effects.

The parenteral formulation of zileuton provided herein would be advantageous for a subject who would benefit from extended use of zileuton. Extended use of zileuton may benefit subjects with acute and serious infections. In some cases, therapeutic options for these subjects can be limited due to multidrug resistance, and polymyxins or aminoglycosides are used as last resort treatment. The pharmaceutical formulation provided herein, which is suitable for the parenteral route of drug administration (for example as an intravenous infusion), may be useful for these subjects, because some may be critically ill and hospitalized, and under these dire circumstances, the oral route is not reliable or feasible for systemic delivery of zileuton to achieve a renal protective effect. In certain examples, the accumulation of zileuton in the kidney (for example proximal tubule epithelia) is increased by parenteral administration of the parenteral zileuton composition by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (for example by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), for example increased by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more as compared to oral administration of the same or equivalent zileuton composition. In certain examples, renal damage or associated symptoms are reduced by parenteral administration of the parenteral zileuton composition by about 10-100%, 20-100%, 30-100%, 40-100%, 50-100%, 60-100%, 70-100%, 80-100%, 20-90%, 30-90%, 40-90%, 50-90%, 60-90%, or 70-90% (for example by about 10-20%, 20-30%, 30-40%, 40-50%, 50-60%, 60-70%, 70-80%, 80-90%, or 90-100%), for example by about 10%, 15%, 20%, 25%, 30%, 35%, 40%, 45%, 50%, 55%, 60%, 65%, 70%, 75%, 80%, 85%, 90%, 95%, or 100%, or at least 10%, 15%, 20%, 25%, 30%, 35%, 40%, 45%, 50%, 55%, 60%, 65%, 70%, 75%, 80%, 85%, 90%, 95%, or 100%, as compared to oral administration of the same or equivalent zileuton composition. As shown in FIGS. 17A-17C, repeated daily intraperitoneal administration of 12 mg/kg zileuton to rats for 10 days was well tolerated, with no significant changes in body weight, serum ALT, or serum creatinine, supporting the safety of extended parenteral use.

The zileuton compositions provided herein can be used as nephroprotective agents to prevent or reduce acute kidney injury across multiple clinical settings. Potential clinical applications include both drug-related and ischemia-related causes of acute kidney injury.

Drug-related acute kidney injury. Zileuton may be administered as an adjunctive therapy to prevent or reduce nephrotoxicity associated with antimicrobial agents (for example aminoglycosides, polymyxins, vancomycin, amphotericin B), chemotherapy (for example cisplatin, methotrexate, ifosfamide), and diagnostic contrast media (for example iodinated contrast agents used in computed tomography and angiography).

Ischemia-related acute kidney injury. Zileuton may be administered prophylactically to prevent or reduce acute kidney injury associated with ischemic shock (for example septic shock, hemorrhagic shock, cardiogenic shock), post-cardiac surgery (for example coronary artery bypass grafting, valve replacement, aortic surgery), and transplant organ procurement (for example deceased donor kidney preservation, living donor nephrectomy).

Methods of preventing or reducing acute kidney injury associated with ischemia-reperfusion are provided. In certain examples, zileuton or a pharmaceutically acceptable salt thereof is administered parenterally to a subject at risk of renal ischemia, including subjects undergoing cardiac surgery, vascular surgery, or organ transplantation, or experiencing ischemic shock. In exemplary embodiments, zileuton is administered at 6 mg/kg as three doses given approximately 10 hours and 2 hours prior to the anticipated ischemia and 6 hours after reperfusion.

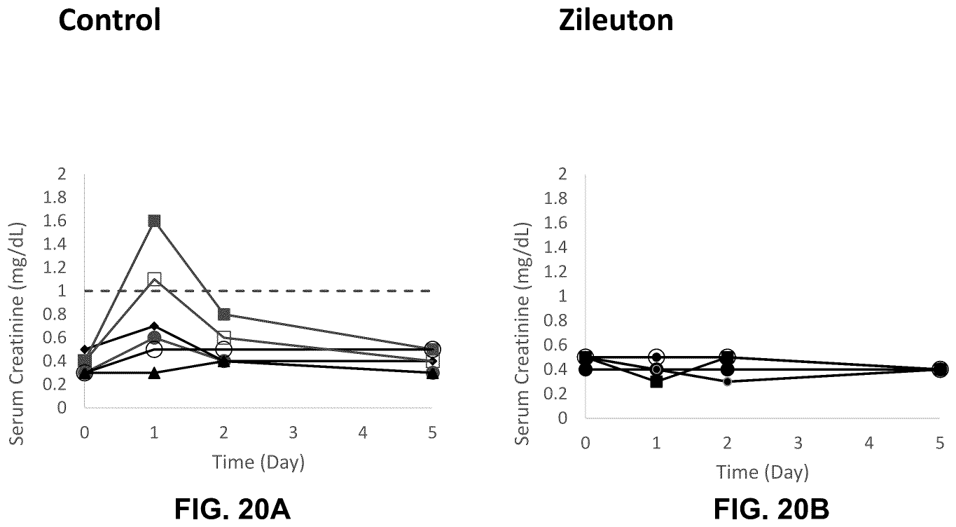

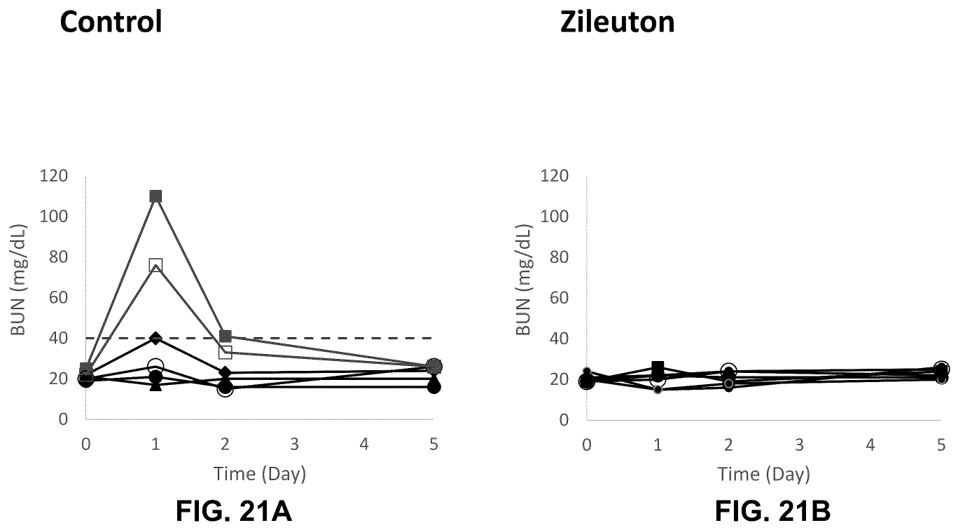

In some embodiments, renal function is monitored by serial measurement of serum creatinine and blood urea nitrogen (BUN) at baseline and at defined intervals after the ischemic insult. An AKI endpoint can be defined as at least a two-fold elevation of the baseline serum creatinine value. As shown in FIGS. 20A-20B and 21A-21B, zileuton administration attenuated post-ischemic increases in serum creatinine and BUN compared with controls.

In certain embodiments, zileuton is administered to prevent or reduce contrast-associated AKI in patients undergoing diagnostic or interventional procedures involving iodinated contrast agents. Prophylaxis can include administration of zileuton prior to and following contrast exposure, for example, within 12 hours before and within 6-12 hours after the procedure, with dosing adjusted based on route of administration and patient-specific factors.

In certain transplantation-related embodiments, zileuton can be included in donor organ preservation or perfusion solutions, or administered to donors or recipients perioperatively, to reduce ischemia-reperfusion injury during procurement and implantation.

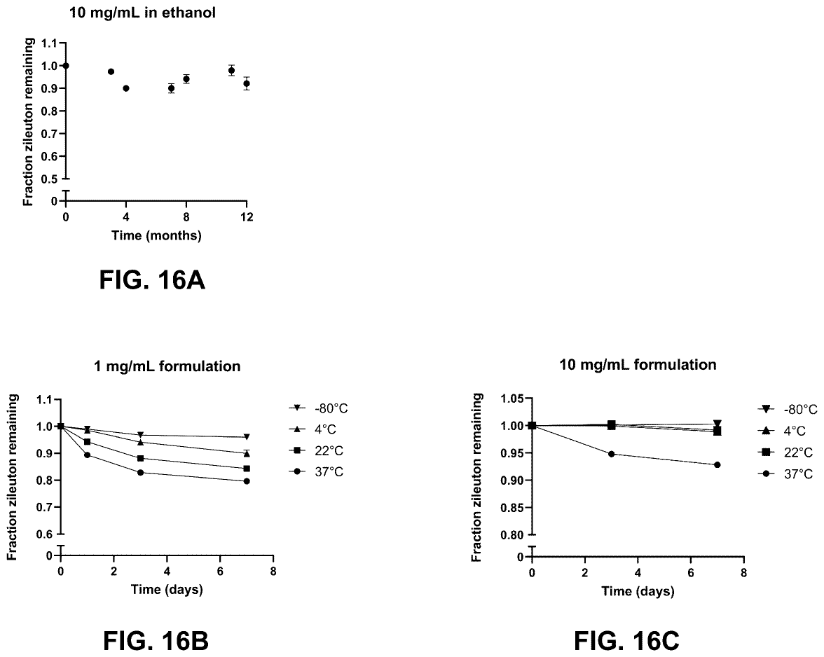

Formulation selection for clinical use can be guided by the expanded ternary solvent solubility mapping shown in FIGS. 15A-15B, selecting compositions that achieve target zileuton concentrations while conforming to parenteral excipient safety parameters, injection tolerability, and stability requirements demonstrated in FIG. 4 and FIGS. 16A-16C.

The following examples are provided to illustrate further aspects associated with the present disclosure, but should not be construed as limiting the scope thereof. Unless otherwise noted, all parts and percentages are by dry weight.

### EXAMPLES

### Example 1: Development of Co-Solvent Parenteral Formulation of Zileuton

Zileuton (MW=236) has a log P value of 0.9 and is practically insoluble in water (<0.5 mg/mL). It is commercially formulated as tablets for oral administration. Here, a co-solvent parenteral formulation for zileuton was developed using FDA-approved excipients:ethanol, PEG 400, and water. In an example, the co-solvents are present in the ratio of 1:4:5 or 10%:25%:65% by volume. The formulation can generate a 1-10 mg/mL zileuton injectable solution to support preclinical and clinical uses. For example, zileuton can first be dissolved in ethanol as a solution of 10 mg/mL and stored at −20° C. Immediately prior to dosing, 0.1 mL of the ethanol solution can be diluted 1:9 with PEG 400:water (4:5 co-solvent) to provide a 1 mg/mL final concentration of zileuton. A zileuton solution (1 mg/mL) was prepared for testing in a rodent model. Based on the target dose (i.e., animal weight), the injection volume can be adjusted accordingly and has ranged from 1 mL to 3 mL given intraperitoneally.

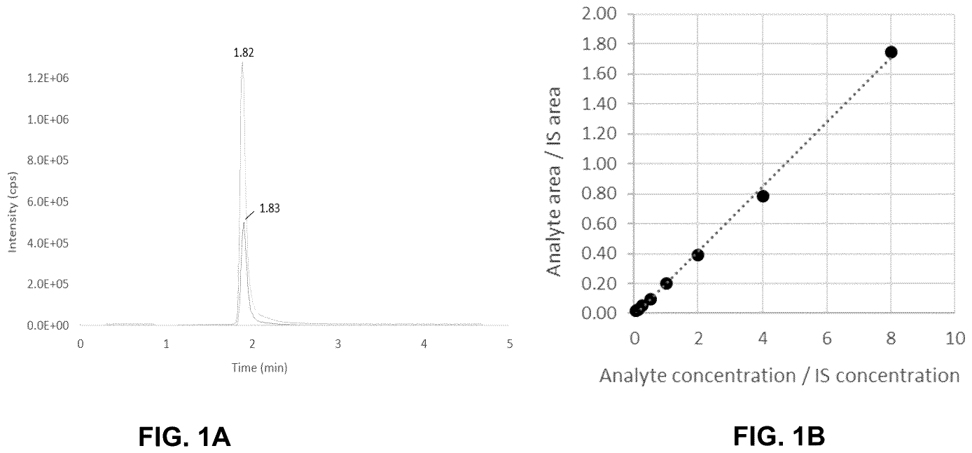

As shown in FIGS. 1A-1B, a liquid chromatography-based (LC-MS/MS) assay to quantify zileuton in aqueous medium and biological samples (for example serum, cell lysate, and kidney tissue homogenate) was developed.

The stable aqueous-based liquid formulation of zileuton is further developed, using FDA-approved water miscible solubilizing solvents, such as polyethylene glycol (PEG 400-600), propylene glycol, glycerin, ethanol, sorbitol, and combinations thereof. A Central Composite Design (CCD), a widely used approach for systematic design of formulation experiments (Aziz et al. 2018 Curr Drug Deliv 15:1330-42; Hassan et al. 2021 Molecules 26), is employed to develop the composition of co-solvents required to solubilize zileuton (10-100 mg) with minimum co-solvents. The formulation candidates are screened based on maximum drug solubility, stability, and blood compatibility. The top three (3) most solubilizing excipients in a co-solvent system are further refined based on a three-factor, five-level rotatable CCD matrix with 20 compositions representing 6 center points, 6 axial points, and 8 edge points of surface designs using the Design-Expert^{®}software.

As shown in FIG. 1A, the analytes were separated by reverse-phase liquid chromatography and detected based on retention times and mass/charge ratios, using multiple reaction monitoring (MRM) scan type in the positive-ion mode. Zileuton-d4 was used as the internal standard (shown in blue on the chromatogram). As shown in FIG. 1B, the linear range of the assay was 0.0625-8 μg/mL zileuton in rat serum (r²=0.997). All co-solvent formulation compositions are tested for zileuton content by LC-MS/MS (see FIGS. 1A-1B), and for color, appearance (for example, precipitation and clarity), pH, in vitro hemolytic potential, and stability at different storage temperatures (for example, 4, 25, 37, 60° C.).

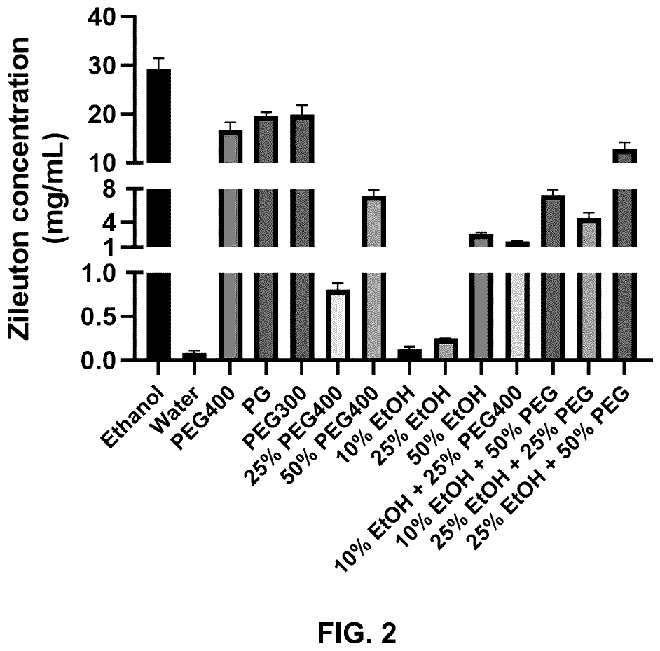

FIG. 2 depicts the solubility of zileuton in various solvents generally regarded as safe (GRAS) by the FDA for parenteral pharmaceutical formulation. FIG. 3 presents a ternary diagram of solubility for zileuton (1 mg/ml). As shown in FIGS. 2 and 3, zileuton is soluble in ethanol or PEG 400, and much less soluble in water. Formulation of zileuton can be adjusted by combining ethanol, PEG 400, and/or water as solvents. In FIG. 3, data for an example formulation according to an embodiment is shown by a star (65% water, 25% PEG 400, 10% ethanol). As shown in FIG. 4, the example zileuton formulation (65% water, 25% PEG 400, 10% ethanol) is stable for over 142 hours at −20, 4, and 22° C., showing no significant degradation.

The long-term (shelf) stability of zileuton formulations was evaluated to support clinical and commercial development. As shown in FIGS. 16A-16C, stability data were generated for zileuton formulations at different concentrations and solvent compositions. FIG. 16A depicts the stability of 10 mg/ml zileuton in ethanol. FIG. 16B depicts the stability of 1 mg/ml zileuton in the ternary co-solvent system (65% water, 25% PEG 400, 10% ethanol). FIG. 16C depicts the stability of 10 mg/ml zileuton in the same ternary co-solvent system (25% water, 50% PEG 400, 25% ethanol).

The data demonstrate that zileuton formulations maintain acceptable stability across a range of concentrations and solvent compositions suitable for parenteral administration. The ethanol stock solution (10 mg/ml) provides a stable concentrated form for storage for over 12 months, while the ternary co-solvent formulations at both 1 mg/ml and 10 mg/ml concentrations exhibit stability profiles compatible with clinical use. These findings support the feasibility of developing stable parenteral zileuton formulations for commercial manufacture and distribution.

### Example 2: In Vivo Evaluation of Co-Solvent Parenteral Formulation of Zileuton

Co-solvent formulations that are safe, non-hemolytic, and effective for parenteral administration are developed as follows.

Up to five (5) top performing co-solvent formulations identified in Example 1 are further evaluated for systemic pharmacokinetics and biodistribution of zileuton after intravenous administration of the suitable formulation in a rodent model. Groups of ten (10) rats (n=5 per gender) are given escalating doses of a single intravenous injection of zileuton (USP). Specific doses to be used are guided by pilot tolerance studies. Blood samples are collected serially, urine/bile samples collected continuously in aliquots, and the kidneys are harvested at selected time points (for example 3, 6, 12, 24 h) after an IV injection of zileuton. Zileuton concentrations in these samples are determined using a validated LC-MS/MS assay (as shown in FIGS. 1A-1B). Based on these concentrations, standard pharmacokinetic parameters (for example volume of distribution, clearance, etc.) are determined and correlated to the clinical equivalent doses in humans. Special emphasis is paid to biodistribution profiles to assess renal residence time and metabolic pathway(s) to evaluate drug-drug interactions. The steady state pharmacokinetics of zileuton is also examined after multiple doses.

To ascertain inhibition of in vivo antibiotic uptake by parenteral administration of zileuton, groups of ten (10) rats (n=5 per gender) are given a single intravenous injection of polymyxin B (USP), with or without the co-administration of zileuton 30 minutes prior. Specific drug doses to be used mimic clinically relevant dosing in humans. Blood samples are collected serially, and the kidneys are harvested at selected time points (for example 3, 6, 24 h) to determine polymyxin B concentrations in the blood or tissue, as described previously (see, for example, Manchandani et al. 2017 Antimicrob Agents Chemother 61:e02391-6). A reduction of >50% in polymyxin B concentrations in renal tissue in zileuton-administered animals as compared to a control without zileuton administration is considered a desirable therapeutic effect of zileuton. Similar studies are performed with amikacin (USP).

FIGS. 5A-5D depict pharmacokinetic profiles of zileuton in serum (FIG. 5A: 4 mg/kg, FIG. 5C: 12 mg/kg) and renal tissue (FIG. 5B: 4 mg/kg, FIG. 5D: 12 mg/kg) in rats following single dose administration. Based on proportional serum area under the curve (AUC), the dosing exposure of 4 mg/kg was estimated to be equivalent to a human dose of approximately 750 mg, and 12 mg/kg was estimated to be equivalent to a human dose of approximately 2250 mg.

To support clinical translation of the zileuton formulation, further comparative pharmacokinetic studies were conducted across multiple routes of administration. Exposure and relative bioavailability of zileuton across administration routes at a 4 mg/kg dose are summarized in FIGS. 18A-18B. As shown in FIG. 18A, mean area under the concentration-time curve (AUC) values for intraperitoneal (IP), oral (PO), and intravenous (IV) dosing were 24, 8, and 32 μg·h/mL, respectively. As shown in FIG. 18B, the corresponding bioavailability estimates for IP, PO, and IV dosing were 75%, approximately 25%, and 100%, respectively. These data indicate that intraperitoneal administration achieves substantially higher bioavailability than oral dosing, approaching that of intravenous administration, and support the use of parenteral formulations to achieve reliable systemic exposure.

Based on the cross-route AUC relationships established in FIGS. 18A-18B, human dose projections were developed. As shown in FIG. 19, the projection relates rat intraperitoneal doses of zileuton to putative human oral and intravenous doses expected to yield comparable systemic exposure. The projections are anchored to the known clinical daily oral dose of 2,400 mg (ZYFLO®) and provide illustrative matched human IV dose estimates under the stated pharmacokinetic assumptions.

Pharmacokinetics of zileuton at steady state was investigated. To provide steady state, zileuton (12 mg/kg) in an example formulation (in 65% water, 25% PEG 400, 10% ethanol) was given once daily for 10 days. As shown in FIG. 6, compared to the first dose, no significant increase in systemic exposure was observed at steady state. These data support dose selection for future clinical development of parenteral zileuton formulations.

### Example 3: Zileuton Ameliorates Aminoglycoside and Polymyxin-Associated Acute Kidney Injury In Vivo

The effect of zileuton in ameliorating the nephrotoxicity of antibiotics (for example amikacin, polymyxin B) was studied in rats. As shown in FIGS. 2 and 3, the aqueous solubility of zileuton is poor (0.14 mg/mL). For initial testing against amikacin, zileuton was first dissolved in DMSO (5 mg/mL) and diluted 5× with phosphate buffered saline prior to administration. For subsequent testing against polymyxin B, a zileuton formulation was developed to facilitate administration of a higher dose. A ternary solvent system consisting of 65% water, 25% PEG 400, and 10% ethanol was used to solubilize zileuton to 1 mg/mL.

The zileuton formulation was well-tolerated by the animals. No gross irritation or evidence of drug precipitation was observed in the intraperitoneal cavity following euthanasia. Additionally, the absorbance of zileuton at 230 nm was used to confirm stability in the formulation for at least 24 hours at room temperature.

The concentration-time profiles of both doses were reasonably characterized (r²>0.99). The AUC_{0-∞} observed with 4 mg/kg and 10 mg/kg zileuton was found to be 27.2 and 93.5 mg*h/L, respectively. The systemic exposure (mean AUC) following administration of zileuton 600 mg was reported to be 19.2 mg. h/L (package insert-ZYFLO®). Based on proportional AUC, the equivalent doses in humans would be 850 mg and 2,922 mg daily, respectively.

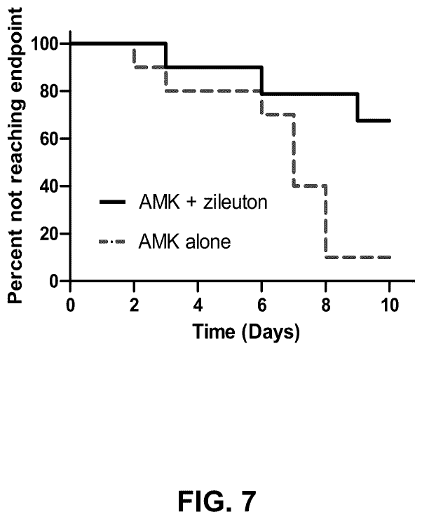

In the amikacin only group, 90% of the animals developed nephrotoxicity within 10 days. The median time to develop nephrotoxicity was 7 days. As shown in FIG. 7, zileuton (4 mg/kg) delivered prior to amikacin administration significantly delayed the onset of nephrotoxicity (p=0.015). In this group, only 30% of the animals developed nephrotoxicity.

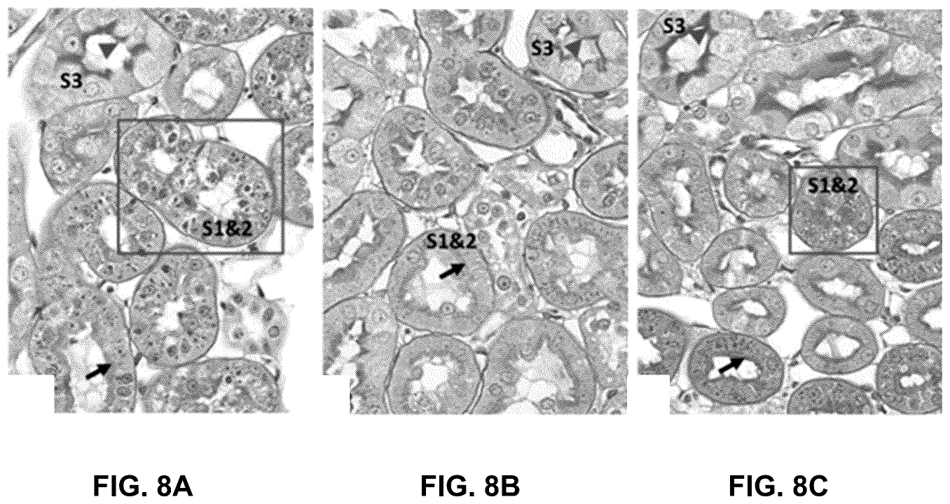

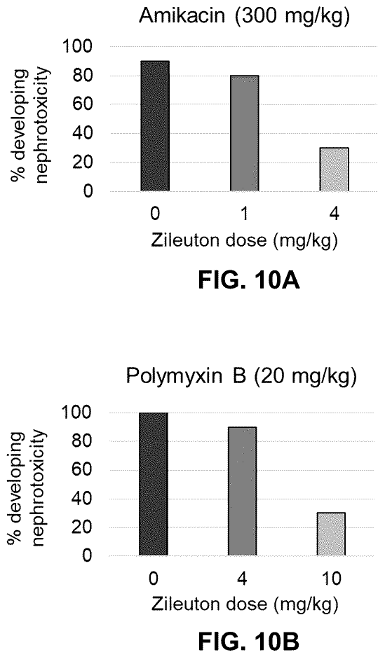

Additionally, as shown in FIG. 8A, histological analysis of kidney sections confirmed significant injury to the proximal tubules in animals treated with amikacin alone. Marked cytoplasmic inclusions, tubular cell injury, and loss of brush border (square), involving the S1 and S2 portions of proximal tubules were observed. A preserved brush border was observed in a less affected proximal tubular profile (FIG. 8A, arrow). The S3 portion of proximal tubules was mostly intact (S3), showing a prominent brush border (FIG. 8A, green arrowhead). As shown in FIG. 8B, in vehicle-treated animals, all portions (FIG. 8B, S1&2, and 3) of proximal tubules were intact without significant changes. The brush border was intact (FIG. 8B, arrow and arrowhead). As shown in FIG. 8C, in animals treated with amikacin and zileuton, less injury was observed as compared with animals treated with amikacin alone. Mild cytoplasmic inclusions and vacuolization involving a few S1&2 profiles were observed (FIG. 8C, square). Most tubular profiles were intact with preservation of the brush border for both S1&2 (FIG. 8C, arrow) and S3 portion (FIG. 8C, arrowhead). In sum, zileuton considerably reduced kidney injury with only mild damage observed in the S1 and S2 portions of the proximal tubules. As shown in FIG. 10A, the percentage of animals developing amikacin-associated nephrotoxicity was reduced in a dose-dependent matter by co-administration of zileuton.

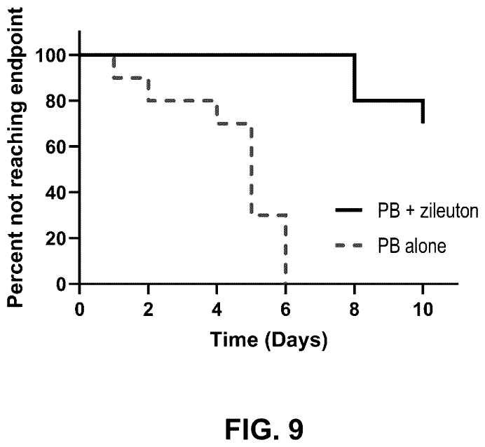

As shown in FIG. 9, in the polymyxin B only group, 100% of the animals developed nephrotoxicity within 10 days. The median time to develop nephrotoxicity was 5 days. Zileuton did not significantly delay nephrotoxicity associated with polymyxin B at 4 mg/kg. As shown in FIG. 9, with a higher dose (10 mg/kg), zileuton significantly delayed the onset of nephrotoxicity (p<0.001). As shown in FIG. 10B, the percentage of animals developing amikacin-associated nephrotoxicity was reduced in a dose-dependent matter by co-administration of zileuton. Only 30% of animals in the group receiving concomitant zileuton developed nephrotoxicity within 10 days.

Dose-limiting nephrotoxicity is the major barrier preventing the optimal clinical use of aminoglycosides and polymyxins. The mechanism of toxicity is widely attributed to reabsorption of these antibiotics from glomerular filtrate by proximal tubule cells via megalin/PEPT2-mediated transport. The exact intracellular pathway is not completely understood, but once inside renal cells, aminoglycosides/polymyxins accumulate to induce mitochondrial dysfunction, reactive oxygen species (ROS) generation, and caspase activation, resulting in apoptosis. This ultimately leads to major degeneration in the kidney cortex resulting in loss of the brush border, increased vascular resistance, and reduced renal blood flow.

Two major therapeutic approaches have been attempted to attenuate nephrotoxicity: (a) reduce antibiotic uptake into proximal tubule cells, or (b) reduce cellular injury following uptake. Reducing drug uptake is a promising approach to attenuate AKI. Anti-inflammatories, mitochondria-acting agents, and antioxidants have also shown potential to reduce cellular injury following aminoglycoside/polymyxin uptake. In contrast to drug uptake inhibition, anti-inflammatories attenuate nephrotoxicity by improving renal perfusion, reducing histological abnormalities, and decreasing the migration of inflammatory cells.

To deliver zileuton parenterally, the present disclosure provides ternary co-solvent formulations of zileuton to overcome its poor solubility in water. The co-solvent formulation achieved a greater than 20-fold increase in aqueous solubility with no apparent side effects. Of note, zileuton had no intrinsic antibacterial activity and it did not interfere with in vitro antibacterial activity or in vivo efficacy of the polymyxins. The data demonstrate zileuton's ability to attenuate nephrotoxicity associated with aminoglycosides and polymyxins in vivo. The onset of nephrotoxicity associated with amikacin and polymyxin B was significantly delayed by concomitant zileuton administration. Furthermore, the overall rates of nephrotoxicity were reduced from 90% (amikacin) and 100% (polymyxin B) to 30% with adjuvant zileuton. Additionally, histopathology confirmed reduced amikacin-associated renal injury in animals receiving zileuton. Specifically, amikacin alone resulted in significant tubular cell injury and loss of the brush border, while amikacin given in combination with zileuton resulted in mostly intact tubular profiles and preservation of the brush border. A trend in dose-response was observed for both antibiotics.

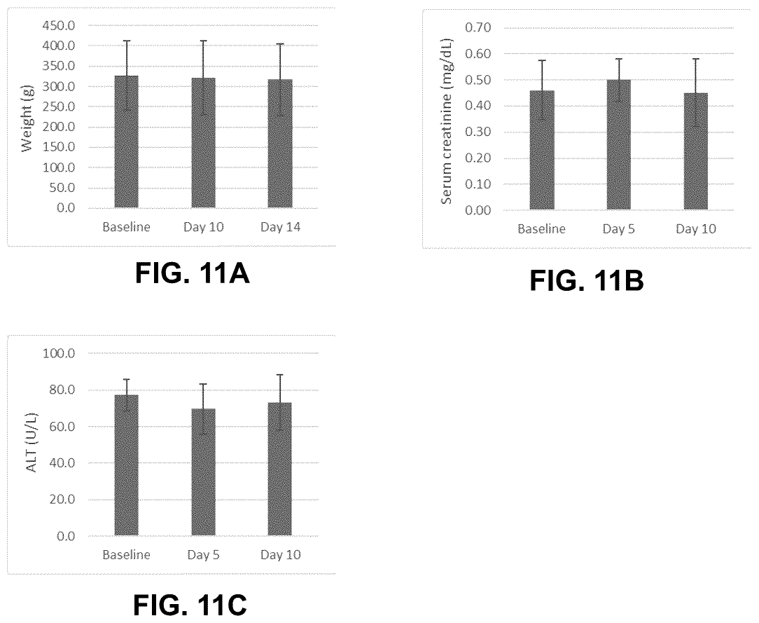

Furthermore, the multi-dose safety of zileuton was confirmed. Zileuton 12 mg/kg was given once daily as the prototype formulation for 10 days (alone without nephrotoxic antibiotics). FIGS. 11A-11C are graphical representations of the multi-dose safety of zileuton. Zileuton 12 mg/kg in an example formulation (in 65% water, 25% PEG 400, 10% ethanol) was administered to rats once daily for 10 days (days 1-10), and weight (FIG. 11A), serum creatinine (FIG. 11B), and ALT (FIG. 11C) were measured at baseline, day 5, day 10, or day 14 as indicated. Compared to baseline, there was no significant change in serum ALT (hepatocellular toxicity marker), serum creatinine (renal function marker), and body weight as observed.

### Example 4: Selective Kidney Accumulation of Nanoparticles in the Kidneys of Mice Without Toxicity

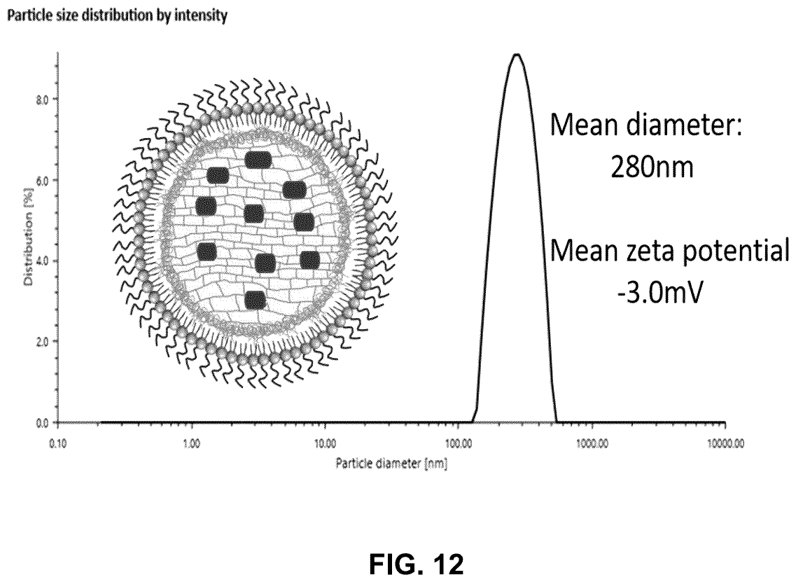

Lipid-polymer hybrid polyplex nanoparticles (NPs) composed of polysaccharide, pegylated lipids, and phosphate counter ions were developed, as schematically depicted in FIG. 12. The NPs have a mean hydrodynamic diameter of 280 nm and near neutral charges (−3 mV). The NPs were designed to have a bigger colloidal particle size, so that the NPs are not subject to kidney filtration into the urine (molecules with a diameter smaller than 10 nm fall below the kidney filtration threshold and can pass through the glomerulus and be excreted into urine quickly). The NP compositions used herein are biomaterials known to be biodegradable in vivo into biologically benign components.

A near infrared fluorescent dye Alexa Fluor 750 (AF750) was conjugated to the NPs and in vivo whole-body imaging was performed in normal nude mice after intravenous (IV) administration. Fluorescence intensity was monitored for up to 3 days. The free unconjugated AF750 control was quickly cleared out of the mice within a few hours (data not shown). On the other hand, as shown in FIG. 13A, the real-time whole animal imaging data showed that kidneys displayed the highest accumulation of AF750-labeled NPs after two (2) hours post-administration, and remained visualized after 2-3 days without apparent toxicity. As shown in FIG. 13B, ex vivo imaging showed more NP accumulation in the kidneys than in the liver or the spleen three (3) days post NP injection. As shown in FIG. 13C, multispectral optoacoustic tomography (MOST) imaging also demonstrated strong kidney accumulation of NPs. These data demonstrate that the NPs have a unique kidney-homing ability when injected in vivo.

### Example 5: Tissue and Cellular Localization of Nanoparticles in Proximal Tubule Epithelium of the Kidney

Three (3) days after an IV injection of AF750-labeled NPs as described in Example 4, anesthetized mice were systemically perfused with PBS buffer. Mice were euthanized, and the kidneys were harvested. Frozen kidneys were sectioned to allow examination of NP localization. FIGS. 14A-14D depict fluorescence images (40×) of kidney after 3 days post IV administration of AF750-labeled NPs in mice. Kidneys were stained with anti-mouse megalin antibody and cell nuclei were stained with DAPI (Red: NPs (FIG. 14A), Green: megalin (FIG. 14B), Blue: nuclei (FIG. 14A), and all overlaid in FIG. 14D) (n=3). As shown in FIGS. 14A-14D, when the tissue sections were counterstained with DAPI, the NPs (red) were found to largely accumulate in the kidneys. The NPs (red) were visualized mainly in close proximity of the megalin-positive renal tubular epithelial cells (green), indicating that the NPs are likely endocytosed into renal tubules via the megalin receptor.

### Example 6: Development of Nanoparticles for Targeted Delivery to the Site of Injury

To enhance delivery of a drug of interest (for example zileuton) to the site of injury, nanoparticles using lipids and polysaccharides as major components, as shown in FIG. 12, are developed. The materials composing the nanoparticles are known to be biocompatible and biodegradable in vivo. Cargo (i.e., zileuton) is incorporated in the inner cavity of the nanoparticles. Alternatively, cargo can be conjugated externally to the nanoparticles. In some cases, modified chitosan is incorporated, which can form nanoparticles by interacting with the ionic cross-linking phosphate salt derivatives via the ionic gelation method (Koukaras et al. 2012 Mol Pharm 9:2856-62; Sacco et al. 2016 Int J Biol Macromol 92:476-83). The amphoteric lipids are further added to generate more compact hybrid nanoparticles via an electrospraying technique. Pharmacokinetic investigations similar to those described in Example 2 are undertaken with various zileuton-nanoparticle formulation(s) including the hybrid nanoparticles described in Example 3 (having a mean hydrodynamic diameter of 280 nm and near neutral charges). An increase in zileuton area under the concentration-time curve (AUC₂₄) in renal tissue of >50% is considered a desirable delivery.

The interaction of NPs with the kidney is size-dependent, owing to the unique ultrastructure of kidney anatomy at the micro-and nano-scales. If the nanoparticles are too small (<6 nm), they can easily cross the glomerular filtration barriers and be excreted in the urine. On the other hand, if the nanoparticles are too big (~500 nm), they can accumulate and are cleared by organs of the reticuloendothelial system (RES). Fine tuning the size of nanoparticles to allow targeting of nanoparticles in the kidneys, while minimizing exposure in other organs, is important for using zileuton nanoparticles as a renal protectant. The ratio and concentration of the polysaccharide, lipids, and counter ions are developed to generate various nanoparticles with a size range of 50-250 nm. Their kidney tubular accumulations are evaluated. Based on results of a systematic investigation of the charge effect of NPs on kidney accumulation, suitable NPs with high kidney accumulation without toxicity are generated. Dynamic light scattering (DLS) and nanoparticle tracking analysis (NTA) techniques are utilized to determine the size as well as the zeta potential of the NPs. The in vivo imaging method is described in FIGS. 13A-13C and Example 4 is used to determine the optimal size and surface charge of NPs that render the highest kidney accumulation with the lowest RES uptake. Additionally, the standard endotoxin contamination assay, microbial contamination assay, in vitro hemolysis assay, in vitro complement activation assay, and stability assay are conducted to evaluate the therapeutic effects of the NP-zileuton formulation.

### Example 7: Extended Solubility Development of Zileuton Co-Solvent Formulations

Building upon the initial formulation development described in Example 1, a systematic solubility screen was performed to further develop the zileuton co-solvent formulation. As shown in FIGS. 15A-15B, an expanded matrix of ethanol:PEG 400:water blends was evaluated to identify compositions that maximize zileuton solubility while maintaining suitability for parenteral administration. FIG. 15A presents a tabulated matrix of formulations A-V listing precise component ratios (ethanol, PEG 400, and water) and the corresponding measured solubility of zileuton (mg/mL) for each composition. FIG. 15B provides a graphical visualization of these solubility data, delineating composition regions within the ethanol/PEG 400/water ternary system that achieve optimal drug solubilization.

The expanded solubility data confirmed that increasing the proportion of ethanol and PEG 400 relative to water enhances zileuton solubility, consistent with the lipophilic character of the drug (log P=0.9). However, formulation selection must balance solubility with safety considerations for parenteral use, including limits on ethanol content to avoid injection site irritation and systemic toxicity. The data presented in FIGS. 15A-15B guide the selection of suitable formulations that achieve clinically relevant zileuton concentrations (1-20 mg/mL) while remaining within acceptable excipient limits for intravenous or intraperitoneal administration.

### Example 8: Tolerance and Safety of Zileuton Formulations

The tolerance and safety of repeated parenteral administration of zileuton formulations were evaluated in rats. As shown in FIGS. 17A-17C, zileuton was administered at 12 mg/kg daily by intraperitoneal injection, and key safety parameters were monitored at baseline, day 10, and day 14 post-administration. FIG. 17A depicts body weight measurements over the study period. FIG. 17B depicts serum alanine aminotransferase (ALT) levels, a marker of hepatocellular toxicity. FIG. 17C depicts serum creatinine levels, a marker of renal function.

No significant changes in body weight, serum ALT, or serum creatinine were observed over the 14-day observation period, indicating that repeated daily parenteral administration of zileuton at 12 mg/kg was well tolerated without evidence of hepatotoxicity or nephrotoxicity. These safety data complement the efficacy data presented in Examples 3 and 9 and support the use of zileuton at therapeutically relevant parenteral doses for extended durations as may be required for prophylaxis or treatment of acute kidney injury.

### Example 9: Zileuton Attenuates Renal Ischemia-Reperfusion Injury In Vivo

Beyond antibiotic-associated nephrotoxicity, the nephroprotective potential of zileuton was evaluated in a model of renal ischemia-reperfusion injury (IRI), a clinically relevant model of acute kidney injury (AKI) that mimics injury patterns observed during cardiac surgery, kidney transplantation, and other conditions associated with transient renal hypoperfusion.

Animals and surgical procedure. Sprague-Dawley female rats (weight 225-250 g) were purchased from Envigo (Indianapolis, Indiana, USA). Animals were kept on a 12-hour light/dark cycle and received food and water ad libitum. Each experimental group was comprised of 6 rats. Animals were anesthetized with isoflurane, and ischemia was induced by bilateral clamping of the renal arteries with nontraumatic vascular clips for 30 minutes. During the operation, animals were maintained on a thermostatically controlled surgical table at 38±1° C. under anesthesia. After surgery, animals were returned to cages with free access to food and water.

Treatment groups and sample collection. Rats were divided into two groups: (1) zileuton-treated animals received zileuton (6 mg/kg×3 doses of 1 mg/ml zileuton in 65% water, 25% PEG 400, 10% ethanol) intraperitoneally at 10 hours and 2 hours prior to surgery and 6 hours post-surgery; (2) control animals received normal saline at the same time points. Blood samples (approximately 200 μL) were collected at baseline, and at 1, 2, and 5 days post-surgery. Blood samples were obtained from the tail tip, allowed to clot, and centrifuged at 10,000×g for 10 minutes. Serum was collected and assayed for creatinine and blood urea nitrogen (BUN) concentrations using the Piccolo Xpress Chemistry Analyzer (Abaxis, Union City, CA, USA). The acute kidney injury (AKI) endpoint was defined as ≥2-fold elevation of the baseline serum creatinine value for each animal.

As shown in FIGS. 20A-20B, serum creatinine trajectories following bilateral renal ischemia-reperfusion injury differed between treatment groups. FIG. 20A depicts serum creatinine concentrations over time in control animals receiving normal saline, while FIG. 20B depicts serum creatinine concentrations in animals treated with zileuton. In control animals, serum creatinine increased substantially following ischemia-reperfusion, consistent with significant renal injury. In contrast, zileuton-treated animals demonstrated attenuated increases in serum creatinine, suggesting a nephroprotective effect of zileuton in the setting of ischemia-reperfusion injury.

As shown in FIGS. 21A-21B, blood urea nitrogen (BUN) trajectories in the same bilateral renal ischemia-reperfusion model paralleled the serum creatinine findings. FIG. 21A depicts BUN concentrations over time in control animals, while FIG. 21B depicts BUN concentrations in zileuton-treated animals. Similar to serum creatinine, BUN elevations were attenuated in animals receiving zileuton compared to saline controls, further supporting the nephroprotective effect of zileuton in this model.

Ischemia-reperfusion injury is characterized by an initial ischemic insult followed by restoration of blood flow, which paradoxically exacerbates tissue damage through oxidative stress, inflammation, and activation of cell death pathways. The 5-lipoxygenase (5-LO) pathway has been implicated in the pathogenesis of ischemia-reperfusion injury, as leukotrienes generated by 5-LO contribute to neutrophil recruitment, vasoconstriction, and inflammatory tissue damage. By inhibiting 5-LO, zileuton may attenuate leukotriene-mediated inflammation and reduce the severity of renal injury following ischemia-reperfusion.

These findings extend the nephroprotective applications of zileuton beyond drug-induced nephrotoxicity to include ischemia-reperfusion injury, a common cause of AKI in hospitalized patients. The data support further investigation of zileuton as a prophylactic agent in clinical settings where renal ischemia-reperfusion is anticipated, such as major cardiovascular surgery or kidney transplantation.

### Materials and Methods

Chemicals and Reagents. Amikacin sulfate (USP) was purchased from Sagent Pharmaceuticals (Schaumburg, Illinois, USA). Polymyxin B (USP) was obtained from Auromedics Pharma (East Windsor, New Jersey, USA). Sterile water and saline were obtained from Covetrus (Portland, Maine, USA). Polyethylene glycol 400 was purchased from Spectrum Chemical (New Brunswick, New Jersey, USA). Ethyl alcohol was obtained from Sigma-Aldrich (St. Louis, Missouri, USA). Zileuton powder (USP) was purchased from Supelco (Bellefonte, Pennsylvania, USA). Piccolo comprehensive metabolic panels were obtained from Abaxis (Union City, California, USA).

Animals. Sprague-Dawley male (weight 325-350 g) and female (weight 225-250 g) rats were obtained from Envigo (Indianapolis, Indiana, USA). Animals were kept on a 12-hour light/dark cycle and received food and water ad libitum. All protocols were approved by the Institutional Animal Care and Use Committee of the University of Houston.

Rat Model of Acute Kidney Injury. Each experimental group was comprised of 10 rats (5 males and 5 females). Animals were administered an antibiotic by subcutaneous injection as previously described (Chan K, et al. Antimicrob Agents Chemother 2020; 64: e00859-20; Manchandani P et al. Antimicrob Agents Chemother 2017; 61: e02391-16). Zileuton (4 mg/kg and 10 mg/kg once daily) was given intraperitoneally, 15 minutes prior to antibiotic administration. Blood samples (approximately 200 μL) were collected at baseline and daily from the tail tip, allowed to clot, and centrifuged at 10,000×g for 10 minutes. The serum was collected and assayed for creatinine concentration by the Piccolo Xpress Chemistry Analyzer (Abaxis, Union City, CA, USA). The endpoint was defined as ≥2-fold elevation of the baseline serum creatinine value for each animal. The onset of nephrotoxicity in different treatment cohorts was compared using a time-to-event (for example Kaplan-Meier) analysis and log rank test. Right censoring was used if the study endpoint was not directly observed within the study timeframe. A p value of <0.05 was considered significant. Kidneys were collected from selected animals for histological examination to characterize the renal injury observed.

As reference controls, amikacin and polymyxin B were given subcutaneously once daily over 10 days. Selection of the amikacin (300 mg/kg) and polymyxin B (20 mg/kg) doses was based on prior pharmacokinetic studies to achieve a total daily drug exposure comparable to that in adults after standard doses.

Kidney histological examination. Kidney sections were examined by light microscopy to assess the extent of kidney injury associated with amikacin exposure. Animals were administered either amikacin only, amikacin and zileuton, or the formulation vehicle only for 3 days. To preserve the kidney tissue, whole-body perfusion-fixation with 10% formalin was conducted. The animals were anesthetized with ketamine/xylazine and perfused with phosphate-buffered saline intracardially using a hydraulic pump (set to 20 mL/min) to flush out the blood. The body was then perfused with 10% formalin for approximately 20 minutes. Both kidneys were removed, fixed in 10% formalin, and stored at 4° C. until analysis. Periodic acid-Schiff reagent was used to stain 1 mm thick sections of the kidneys, which were then examined under a light microscope.

Pharmacokinetic studies. Three male and three female rats were administered zileuton (single doses of 4 and 10 mg/kg) intraperitoneally and serial blood samples (approximately 100 μL each) were taken over 12 hours from the tail tip. The blood samples were allowed to clot at room temperature, centrifuged at 10,000×g for 10 minutes, and the serum was collected and stored at −20° C. until analysis. Following quantification, the serum concentrations of each time point were averaged, and the time-profile was characterized using a 1-compartment model with first order absorption in ADAPT 5 (University of Southern California, Los Angeles, CA, USA). AUC_{0-∞} was derived by dividing the dose by the best-fit clearance estimate.

Drug assay. An ultra-performance liquid chromatography tandem mass spectrometry (LC-MS/MS) method was adapted from previous publications for quantification of zileuton in human plasma (Pian Pet et al. Journal of Chromatography 2013; 937:79-83; Armoudjian Y et al. Journal of Pharmaceutical Innovation 2020; 15:581-90.). Chromatographic separation of zileuton and zileuton-d4 (internal standard) was achieved over 5 minutes using the ExionLC AD UHPLC system from Applied Biosystems/MDS SCIEX (Foster City, CA, USA). A Kinetex® EVO C18 column (100×2.1 mm internal diameter, 5 μm) with column oven temperature of 30° C., injection volume of 2 μL, and flow rate of 0.2 mL/min was used. An isocratic mobile phase consisting of 0.1% formic acid (60%) and 0.1% formic acid in acetonitrile (40%) was used. An API 5500 QTrap Triple-Quadrupole mass spectrometer with a TurboIonSpray™ source from Applied Biosystems/MDS SCIEX was used to accomplish mass spectrophotometric detection. The multiple reaction monitoring (MRM) method was operated in positive-ion mode to determine the best transition pairs for zileuton (m/z 237.3→161.1) and zileuton-d4 (m/z 241.2→165.1), respectively. The Analyst® 1.6.3 software (Foster City, CA, USA) was used to quantify analyte and internal standard concentrations.

Preparation of standards and samples for quantification. Working solutions of zileuton in 2× concentration dilutions from 0.1875 mg/L to 96 mg/L were prepared in 40% methanol, from a 10 mg/mL stock solution in ethanol stored at −20° C. The internal standard solution was prepared by dilution of a stock solution to 20 mg/L. Following preparation of the working solutions, 15 μL of serum was mixed with 5 μL zileuton working solution and 10 μL of zileuton-d4 working solution. Rat serum samples (15 μL) were mixed with 10 μL of internal standard and 5 μL of 40% methanol. Then 300 μL of cold acetonitrile was added, and the mixture was vortexed for 30 seconds followed by centrifugation at 15,000×g for 15 minutes. The supernatant was recovered and dried under a stream of nitrogen. Standards and samples were then reconstituted in 600 μL LCMS-grade water and centrifuged for 15 minutes at 15,000×g. The linear range of the assay was 0.125-32 mg/L and the LLOQ was 0.0625 mg/L. Each calibration curve was calculated using a 1/x weighting of the linear regression. Intra-and interday precision and accuracy were acceptable (CV % and error %<10%). The percent recovery was very good (>85%) and the matrix effect was not significant (<±10%).

Embodiments include a pharmaceutical composition comprising a therapeutically effective amount of zileuton and a biologically acceptable excipient formulated for parenteral administration. In certain examples, the excipient contains water, saline, polyethylene glycol (PEG), propylene glycol, glycerin, ethanol, sorbitol, triacetin, polyoxyethylated glycerides, polyoxyethylated oleic glycerides, hydroxypropyl-beta-cyclodextrin, a surfactant, or any combination thereof. In certain examples, the PEG has a molecular weight of about 350-650 Daltons, such as PEG 400, PEG 500, PEG 600. In certain examples, the surfactant includes one or more of hydroxypropylcellulose 20, polysorbate 80, polysorbate 20, sorbitan monooleate NF, polyoxyl 40 hydrogenated castor oil, and polyoxyl hydroxystearates. In certain examples, the excipient contains ethanol, PEG 400, and water. In certain examples, the excipient contains ethanol, PEG 400, and water in 10%:40%:50% by volume. In certain examples, the excipient contains ethanol, PEG 400, and water in 10%:25%:65% by volume. In certain examples, the excipient includes a nanoparticle comprising a polysaccharide, a pegylated lipid, phosphate counter ions, or any combination of any thereof. In certain examples, the nanoparticle has a mean hydrodynamic diameter of about 50-300 nm and a near neutral charge. In certain examples, the nanoparticle has a mean hydrodynamic diameter of about 280 nm and a mean zeta potential of about −3 mV. In certain examples, the nanoparticle contains zileuton in an inner cavity thereof.

In certain examples, the pharmaceutical composition comprises at least about 1 mg/ml of zileuton. In certain examples, the zileuton degrades less than 10% over 142 hours at −20° C., 4° C., or 22° C. in the pharmaceutical composition.

Embodiments include the pharmaceutical composition provided herein, for use in preventing, delaying the onset of, or treating acute kidney injury or nephrotoxicity in a subject. In certain examples, the AKI or nephrotoxicity is induced by an antibiotic or an anti-cancer drug. In certain examples, the antibiotic is one or more of amikacin, tobramycin, netilmicin, streptomycin, arbekacin, plazomicin, neomycin, kanamycin, paromomycin, gentamicin, bacitracin, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815, and octapeptin C4. In certain examples, the anti-cancer drug is one or more of ifosfamide, ipilimumab, pembrolizumab, and nivolumab.

Embodiments include the pharmaceutical composition provided herein, for intravenous, intraperitoneal, subcutaneous, intramuscular, or transdermal administration to a subject. Embodiments include the pharmaceutical composition provided herein, for parenteral administration to a critically ill subject or a subject having severe infection or infection with multidrug resistant bacteria.

Embodiments include methods for preparing a pharmaceutical composition by combining a therapeutically effective amount of zileuton and a biologically acceptable excipient for parenteral administration. In certain examples, the excipient includes two or more of water, saline, polyethylene glycol (PEG) (such as PEG with a molecular weight of about 350-650 Daltons, PEG 400 , PEG 500, PEG 600), propylene glycol, glycerin, ethanol, sorbitol, triacetin, polyoxyethylated glycerides, polyoxyethylated oleic glycerides, hydroxypropyl-beta-cyclodextrin, a surfactant, or any combination thereof. In certain examples, the surfactant can be one or more of hydroxypropylcellulose 20, polysorbate 80, polysorbate 20, sorbitan monooleate NF, polyoxyl 40 hydrogenated castor oil, and polyoxyl hydroxystearates. In certain examples, the excipient contains ethanol, PEG 400, and water. In certain examples, the excipient contains ethanol, PEG 400, and water in 10%:40%:50% by volume. In certain examples, the excipient contains ethanol, PEG 400, and water in 10%:25%:65% by volume.

In certain examples, the excipient contains a nanoparticle with a polysaccharide, a pegylated lipid, phosphate counter ions, or a combination of any thereof. In certain examples, the nanoparticle has a mean hydrodynamic diameter from about 50 nm to about 300 nm of about 50-300 nm and a near neutral charge. In certain examples, the nanoparticle has a mean hydrodynamic diameter of about 280 nm and a mean zeta potential of about −3 mV.

In certain examples, the method includes loading the zileuton into an inner cavity of the nanoparticle, thereby preparing the pharmaceutical composition for parenteral administration. In certain examples, the pharmaceutical composition comprises at least about 1 mg/ml of zileuton. In certain examples, the zileuton degrades less than 10% at −20° C., 4° C., or 22° C. in the pharmaceutical composition.

Embodiments include a method of preparing the pharmaceutical composition provided herein, for use in preventing, delaying the onset of, or treating acute kidney injury or nephrotoxicity in a subject. In certain examples, the AKI or nephrotoxicity is induced by an antibiotic or an anti-cancer drug. In certain examples, the antibiotic is one or more of amikacin, tobramycin, netilmicin, streptomycin, arbekacin, plazomicin, neomycin, kanamycin, paromomycin, gentamicin, bacitracin, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815, and octapeptin C4. In certain examples, the anti-cancer drug is one or more of ifosfamide, ipilimumab, pembrolizumab, and nivolumab.

Embodiments include a method of preparing the nanoparticle composition for intravenous, intraperitoneal, subcutaneous, intramuscular, or transdermal administration to a subject. Embodiments include a method of preparing the nanoparticle composition for parenteral administration to a critically ill subject or a subject having a severe infection or infection with multidrug resistant bacteria. Embodiments include a method of administering a therapeutically effective amount of zileuton to a subject by administering parenterally to the subject a nanoparticle composition comprising a therapeutically effective amount of zileuton and a biologically safe excipient formulated for parenteral administration. In certain examples, the method includes administering the nanoparticle composition intravenously, intraperitoneally, subcutaneously, intramuscularly, or transdermally to the subject.

In certain examples, the method includes administering the pharmaceutical composition in a single dose to the subject. In certain examples, the method includes administering the pharmaceutical composition in multiple doses, at regular or varied intervals to the subject. In certain examples, the method includes administering the pharmaceutical composition to achieve a serum zileuton concentration of about 1 mg/kg to about 12 mg/kg in the subject. In certain examples, the method includes administering about 880 mg to about 3000 mg of zileuton to the subject. In certain examples, the method includes administering the pharmaceutical composition to a critically ill subject or a subject having a severe infection or infection with multidrug resistant bacteria. In certain examples, the method prevents, delays onset of, or treats acute kidney injury or nephrotoxicity in a subject. In certain examples, the AKI or nephrotoxicity is induced by an antibiotic or an anti-cancer drug.

Embodiments include a method of treating a subject with infection, comprising administering parenterally to the subject a pharmaceutical composition comprising (i) a therapeutically effective amount of zileuton and a biologically acceptable excipient formulated for parenteral administration; and (ii) a therapeutically effective amount of a nephrotoxicity-inducing antibiotic selected from the group consisting of amikacin, tobramycin, netilmicin, streptomycin, arbekacin, plazomicin, neomycin, kanamycin, paromomycin, gentamicin, bacitracin, polymyxin B, colistin, amphotericin B, tetracyclines, polymyxin B sulfate, colistin sulfomethate, colistin methanesulfonate, sodium colistimethate, MRX-8, SPR741, SPR206, CA824, FADDI-002, FADDI-003, FADDI-287, MICuRx-12, NAB739, NAB815, and octapeptin C4.

Embodiments include a method of treating a subject with cancer, comprising administering parenterally to the subject a pharmaceutical composition comprising (i) a therapeutically effective amount of zileuton and a biologically acceptable excipient formulated for parenteral administration; and (ii) a therapeutically effective amount of a nephrotoxicity-inducing anti-cancer drug selected from the group consisting of ifosfamide, ipilimumab, pembrolizumab, and nivolumab. In certain examples, the therapeutically effective amount of zileuton ranges from about 0.5 mg/kg to 200 mg/kg. In certain examples, the therapeutically effective amount of zileuton ranges from about 1 mg/kg to 12 mg/kg. In certain examples, the method includes administering the pharmaceutical composition comprising about 880 mg to about 3000 mg of zileuton to the subject. Embodiments include a kit for parenteral administration of zileuton, including a pharmaceutical composition comprising a therapeutically effective amount of zileuton and a biologically acceptable excipient formulated for parenteral administration, and an instruction for use.

In certain examples, the excipient includes water, saline, polyethylene glycol (PEG), PEG with a molecular weight of about 350-650 Daltons, PEG 400 , PEG 500, PEG 600, propylene glycol, glycerin, ethanol, sorbitol, triacetin, polyoxyethylated glycerides, polyoxyethylated oleic glycerides, hydroxypropyl-beta-cyclodextrin, a surfactant, or any combination thereof. In certain examples, the surfactant can be one or more of hydroxypropylcellulose 20, polysorbate 80, polysorbate 20, sorbitan monooleate NF, polyoxyl 40 hydrogenated castor oil, and polyoxyl hydroxystearates. In certain examples, the excipient includes ethanol, PEG 400 , and water. In certain examples, the excipient contains ethanol, PEG 400, and water in 10%:40%:50% by volume. In certain examples, the excipient contains ethanol, PEG 400, and water in 10%:25%:65% by volume. In certain examples, the excipient includes a nanoparticle comprising a polysaccharide, a pegylated lipid, phosphate counter ions, or a combination of any thereof. In certain examples, the nanoparticle has a mean hydrodynamic diameter of about 50-300 nm and a near neutral charge. In certain examples, the nanoparticle has a mean hydrodynamic diameter of about 280 nm and a mean zeta potential of about −3 mV. In certain examples, the nanoparticle contains zileuton in an inner cavity thereof. In certain examples, the pharmaceutical composition includes at least about 1 mg/mL of zileuton. In certain examples, the zileuton degrades less than 10% over 140-150 hours at −20° C., 4° C., or 22° C. in the pharmaceutical composition. In certain examples, the kit is for preventing, delaying the onset of, or treating acute kidney injury or nephrotoxicity in a subject. In certain examples, the AKI or nephrotoxicity is induced by an antibiotic or an anti-cancer drug. In certain examples, the kit contains an antibiotic, which is one or more of amikacin, tobramycin, netilmicin, streptomycin, arbekacin, plazomicin, neomycin, kanamycin, paromomycin, gentamicin, bacitracin, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815, and octapeptin C4. In certain examples, the kit contains the anti-cancer drug, which is one or more of ifosfamide, ipilimumab, pembrolizumab, and nivolumab. In certain examples, the kit contains the pharmaceutical composition for intravenous, intraperitoneal, subcutaneous, intramuscular, or transdermal administration to a subject. In certain examples, the kit is for parenteral administration of zileuton to a critically ill subject or a subject having severe infection or infection with multidrug resistant bacteria.

Other objects, features, and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A pharmaceutical composition comprising a therapeutically effective amount of zileuton and a biologically acceptable excipient formulated for parenteral administration.

2. The pharmaceutical composition of claim 1, wherein the excipient comprises water, saline, polyethylene glycol (PEG) of molecular weight ranging from about 350-650 Daltons, propylene glycol, glycerin, ethanol, sorbitol, triacetin, polyoxyethylated glycerides, polyoxyethylated oleic glycerides, hydroxypropyl-beta-cyclodextrin, a surfactant, or any combination thereof.

3. The pharmaceutical composition of claim 2, wherein the surfactant comprises one or more of hydroxypropylcellulose 20, polysorbate 80, polysorbate 20, sorbitan monooleate NF, polyoxyl 40 hydrogenated castor oil, and polyoxyl hydroxystearates.

4. The pharmaceutical composition of claim 2, wherein the excipient comprises ethanol, PEG 400, and water.

5. The pharmaceutical composition of claim 4, wherein ethanol is about 10% by volume and PEG 400 is about 25% to about 40% by volume.

6. The pharmaceutical composition of claim 4, wherein the excipient contains ethanol, PEG 400, and water in 10%:40%:50% by volume.

7. The pharmaceutical composition of claim 1, wherein the excipient comprises a nanoparticle comprising a polysaccharide, a pegylated lipid, phosphate counter ions, or any combination thereof.

8. The pharmaceutical composition of claim 7, wherein the nanoparticle has a mean hydrodynamic diameter of about 50-300 nm and a near neutral charge.

9. The pharmaceutical composition of claim 7, wherein the nanoparticle contains zileuton in an inner cavity of the nanoparticle.

10. A method of preventing, delaying onset of, or treating acute kidney injury (AKI) or nephrotoxicity in a subject by parenterally administering a pharmaceutical composition comprising a therapeutically effective amount of zileuton and a biologically acceptable excipient formulated for parenteral administration.

11. The method of claim 10, wherein the AKI or nephrotoxicity is induced by a drug.

12. The method of claim 11, wherein the drug comprises an antibiotic, an anti-cancer drug, or a diagnostic contrast agent.

13. The method of claim 12, wherein the antibiotic is one or more of amikacin, tobramycin, netilmicin, streptomycin, arbekacin, plazomicin, neomycin, kanamycin, paromomycin, gentamicin, bacitracin, colistin, amphotericin B, tetracyclines, polymyxin B, polymyxin B sulfate, colistin sulfomethate, sodium colistimethate, MRX-8, SPR741, SPR206, CA824, FADDI-002, FADDI-003, FADDI-287, MICuRx-12, NAB739, NAB815, and octapeptin C4.

14. The method of claim 12, wherein the anti-cancer drug is one or more of ifosfamide, ipilimumab, pembrolizumab, and nivolumab.

15. The method of claim 11, comprising administering the pharmaceutical composition prior to or contemporaneously with administration of the drug.

16. The method of claim 10, wherein the pharmaceutical composition is administered intravenously, intramuscularly, subcutaneously, transdermally, intraperitoneally, intranasally, or intrathecally.

17. The method of claim 10, wherein the AKI or nephrotoxicity is induced by renal ischemia.

18. The method of claim 17, wherein the renal ischemia is associated with ischemic shock, surgery, or transplant organ procurement.

19. The method of claim 17, wherein the method comprises administering the pharmaceutical composition at about 10 hours and about 2 hours prior to an event that may induce the renal ischemia, and 6 hours following the event.

20. The method of claim 19, wherein the event is ischemic shock, surgery, or transplant organ procurement.
